(19) United States
(12) Patent Application Publication
(10) Pub. No.: US 2023/0389803 A1
(43) Pub. Date: Dec. 7, 2023

(54) Multilayer Smart Bra or Bra Insert for Optical Detection of Breast Cancer

(71) Applicant: Robert A. Connor, St. Paul, MN (US)

(72) Inventor: Robert A. Connor, St. Paul, MN (US)

(73) Assignee: Holovisions LLC, St. Paul, MN (US)

(21) Appl. No.: 18/237,231

(22) Filed: Aug. 23, 2023

Related U.S. Application Data
(63) Continuation-in-part of application No. 18/096,748, filed on Jan. 13, 2023.
Application No. 18/096,748 is a continuation-in-part of application No. 17/897,182, filed on Aug. 28, 2022.
Application No. 17/897,182 is a continuation-in-part of application No. 16/933,138, filed on Jul. 20, 2020, now abandoned.
(60) Provisional application No. 62/879,485, filed on Jul. 28, 2019.

(51) Int. Cl. A61B 5/00; A41C 3/00

(52) U.S. Cl. CPC A61B 5/0091; A61B 5/6804; A61B 5/6843; A61B 5/6844; A41C 3/0064

(57) ABSTRACT

This device is a multi-layer smart bra or bra insert for optical detection of breast cancer. It has four layers: an air-gap-reducing layer; an optical layer with a plurality of light emitters and light detectors; an expandable layer; and a structural layer. Light from the light emitters which has been transmitted through and/or reflected from breast tissue and received by the light detectors is analyzed to detect and/or image abnormal breast tissue.

### CROSS-REFERENCE TO RELATED APPLICATIONS

This application is a continuation-in-part of U.S. application Ser. No. 18/096,748 filed on 2023 Jan. 13. U.S. application Ser. No. 18/096,748 was a continuation-in-part of U.S. application Ser. No. 17/897,182 filed on 2022 Aug. 28. U.S. application Ser. No. 17/897,182 was a continuation-in-part of U.S. application Ser. No. 16/933,138 filed on 2020 Jul. 20. U.S. application Ser. No. 16/933,138 claimed the priority benefit of U.S. provisional application 62/879,485 filed on 2019 Jul. 28. The entire contents of these related applications are incorporated herein by reference.

### FEDERALLY SPONSORED RESEARCH

Not Applicable

### SEQUENCE LISTING OR PROGRAM

Not Applicable

### BACKGROUND—FIELD OF INVENTION

This invention relates to wearable devices for medical imaging and diagnosis.

### INTRODUCTION

Early detection of breast cancer is vital. Current methods for breast cancer detection include x-ray mammography, MRI, and ultrasound. However, these methods have limitations. X-ray mammography causes exposure to ionizing radiation, MRI equipment is expensive and not very mobile, and ultrasound can have low sensitivity. Optical imaging and spectroscopic analysis is another method for breast cancer detection which can address these limitations.

Optical imaging and spectroscopic analysis of breast tissue takes advantage of differences in light absorption and scattering by normal tissue vs. abnormal tissue. There are several biomarkers which are present in different concentrations in abnormal tissue. These biomarkers include deoxygenated hemoglobin, oxygenated hemoglobin, lipids, collagen, oxygen, and water. When near-infrared light is transmitted through and/or reflected from breast tissue, changes in light transmission caused by these biomarkers can be used to detect and image abnormal breast tissue.

With continuous wave (CW) optical analysis methods, light emitters direct light with constant intensity into and/or onto breast tissue. With frequency domain (FD) optical analysis methods, light emitters direct modulated light into and/or onto breast tissue. With time domain (TD) optical analysis methods, light emitters direct pulses of light into and/or onto breast tissue.

Variations on optical imaging and spectroscopic analysis methods in the prior art include: Diffuse Optical Imaging (DOI), Diffuse Optical Spectroscopic Imaging (DOSI), Diffuse Optical Spectroscopy (DOS), Diffuse Optical Tomography (DOT), Frequency-Domain Photon Migration (FDPM), Functional Near-Infrared Spectroscopy (fNIRS), Near-Infrared Spectroscopy (NIRS), Raman spectroscopy, Reflectance Diffuse Optical Tomography (RDOT), Transillumination Imaging (TI), and/or Transmittance Diffuse Optical Tomography (TDOT).

Although optical methods for detection of breast cancer are promising, there some limitations with devices and methods in the prior art. One challenge is the difficulty of using optical methods to scan greater tissue depth because light is increasingly scattered through larger spans of tissue. Another challenge is errors due to air gaps between optical components and the breast surface. There remains a need for innovative optical devices and methods to detect breast cancer which solve these challenges.

### REVIEW OF THE RELEVANT ART

In the patent literature, U.S. patent application 20050043596 (Chance, Feb. 24, 2005, “Optical Examination Device, System and Method”) discloses a brush-form optical coupler with freely extending fiber end portions, sized and positioned to make optical contact with a subject, examination, and monitoring systems utilizing one or more of such couplers. U.S. patent application 20060058683 (Chance, Mar. 16, 2006, “Optical Examination of Biological Tissue Using Non-Contact Irradiation and Detection”) and U.S. Pat. No. 7,904,139 (Chance, Mar. 8, 2011, “Optical Examination of Biological Tissue Using Non-Contact Irradiation and Detection”) disclose an optical system for examination of biological tissue which includes a light source, a light detector, optics and electronics. Sometimes inventions are the result of serendipitous insights; in this case, optical scanning of biological tissue may actually have been invented by chance.

U.S. Pat. No. 6,081,322 (Barbour, Jun. 27, 2000, “NIR Clinical Opti-Scan System”) and RE38800 (Barbour, Sep. 20, 2005, “NIR Clinical Opti-Scan System”) disclose three-dimensional optical imaging techniques for the detection and three-dimensional imaging of absorbing and/or scattering structures in complex random media, such as human body tissue, by detecting scattered light. U.S. patent application 20150182121 (Barbour, Jul. 2, 2015, “Low-Cost Screening System for Breast Cancer Detection”) discloses a portable and wearable tumor detector including a brassier and devices for optical tomography. U.S. patent application publication 20150119665 (Barbour et al., Apr. 30, 2015, “Self-Referencing Optical Measurement for Breast Cancer Detection”) and U.S. Pat. No. 9,724,489 (Barbour et al., Aug. 8, 2017, “Self-Referencing Optical Measurement for Breast Cancer Detection”) disclose obtaining optical data from a pair of breasts, employing a simultaneous bilateral referencing protocol, and employing a self-referencing data analysis method.

U.S. patent applications 20100292569 (Hielscher et al., Nov. 18, 2010, “Systems and Methods for Dynamic Imaging of Tissue Using Digital Optical Tomography”) and 20150223697 (Hielscher et al., Aug. 13, 2015, “Systems and Methods for Dynamic Imaging of Tissue Using Digital Optical Tomography”) disclose methods for imaging tissue using diffuse optical tomography including directing a amplitude modulated optical signals from optical signal sources. U.S. patent application 20140330116 (Hielscher et al., Nov. 6, 2014, “Systems and Methods for Simultaneous Multi-Directional Imaging for Capturing Tomographic Data”) discloses devices, systems, and method for tomographic imaging in which light transmitted and backscattered surface light is imaged by an optical system that minimizes reflection back to the target object. U.S. patent applications 20130289394 (Hielscher et al., Oct. 31, 2013, “Dynamic Optical Tomographic Imaging Devices Methods and Systems”), 20170027480 (Hielscher et al., Feb. 2, 2017, “Dynamic Optical Tomographic Imaging Devices Methods and Systems”), and 20190282134 (Hielscher et al., Sep. 19, 2019, “Dynamic Optical Tomographic Imaging Devices Methods and Systems”), and U.S. patent Ser. No. 10/178,967 (Hielscher et al., Jan. 15, 2019, “Dynamic Optical Tomographic Imaging Devices Methods and Systems”) disclose an optical tomographic systems for acquiring and displaying dynamic data representing changes in a target tissue sample to external provocation. U.S. patent applications 20130338496 (Hielscher et al., Dec. 19, 2013, “Medical Imaging Devices, Methods, and Systems”) and 20140088415 (Hielscher et al., Mar. 27, 2014, “Medical Imaging Devices, Methods, and Systems”) disclose devices, methods, and systems for generating optical tomographic data including volumetric and surface geometric data.

U.S. patent application publication 20140236003 (Hielscher et al., Aug. 21, 2014, “Interfacing Systems, Devices, and Methods for Optical Imaging”) discloses an imaging interface with a plurality of concentric rings for diffuse optical tomography of breast tissue. U.S. patent applications 20140243681 (Hielscher et al., Aug. 28, 2014, “Compact Optical Imaging Devices, Systems, and Methods”) and 20190239751 (Hielscher et al., Aug. 8, 2019, “Compact Optical Imaging Devices, Systems, and Methods”), and U.S. patent Ser. No. 10/111,594 (Hielscher et al., Oct. 30, 2018, “Compact Optical Imaging Devices, Systems, and Methods”) disclose a handheld optical imaging system with a plurality of detectors. U.S. patent application 20150286785 (Hielscher et al., Oct. 8, 2015, “Systems, Methods, and Devices for Image Reconstruction Using Combined PDE-Constrained and Simplified Spherical Harmonics Algorithm”) and U.S. Pat. No. 9,495,516 (Hielscher et al., Nov. 15, 2016, “Systems, Methods, and Devices for Image Reconstruction Using Combined PDE-Constrained and Simplified Spherical Harmonics Algorithm”) disclose systems, methods, and devices for image reconstruction using combined PDE-constrained and simplified spherical harmonics (SPN) algorithms. U.S. patent Ser. No. 10/376,150 (Hielscher et al., Aug. 13, 2019, “Interfacing Systems, Devices, and Methods for Optical Imaging”) discloses an imaging interface for diffuse optical tomography of breast with a plurality of concentric rings.

U.S. patent application publication 20140236021 (Islam, Aug. 21, 2014, “Near-Infrared Super-Continuum Lasers for Early Detection of Breast and Other Cancers”) and U.S. Pat. No. 9,993,159 (Islam, Jun. 12, 2018, “Near-Infrared Super-Continuum Lasers for Early Detection of Breast and Other Cancers”) disclose a system and method using near-infrared or short-wave infrared light sources for early detection and monitoring of breast cancer. U.S. patent application publication 20180289264 (Islam, Oct. 11, 2018, “High Signal-to-Noise Ratio Light Spectroscopy of Tissue”) discloses a diagnostic system which delivers an optical beam to a nonlinear element that broadens a spectrum of the first optical beam to at least 10 nanometers through a nonlinear effect in the nonlinear element. U.S. patent application 20210038083 (Islam, Feb. 11, 2021, “Multi-Wavelength Wearable Device for Non-Invasive Blood Measurements in Tissue”) discloses a system for measuring one or more physiological parameters with a wearable device that includes a light source comprising a driver and semiconductor sources that generate an output optical light.

U.S. patent application publication 20090005692 (Intes et al., Jan. 1, 2009, “Optical Imaging Method for Tissue Characterization”) and U.S. Pat. No. 8,565,862 (Intes et al., Oct. 22, 2013, “Optical Imaging Method for Tissue Characterization”) disclose a method for detecting and characterizing abnormalities within biological tissue by characterizing optical properties of the tissue. U.S. patent application publication 20180070891 (Jepsen, Mar. 15, 2018, “Imaging With Infrared Imaging Signals”) discloses using an infrared imaging signal to image tissue. U.S. patent application publication 20180335753 (Jepsen et al., Nov. 22, 2018, “Co-Located Imaging and Display Pixel”) discloses an optical transformation engine coupled between an image pixel and a display pixel. U.S. patent application publication 20190072897 (Jepsen et al., Mar. 7, 2019, “Applications of Diffuse Medium Imaging”) discloses methods and an apparatus for imaging translucent materials.

U.S. Pat. No. 9,314,218 (Stearns et al., Apr. 19, 2016, “Integrated Microtomography and Optical Imaging Systems”) and 10130318 (Stearns et al., Nov. 20, 2018, “Integrated Microtomography and Optical Imaging Systems”) disclose an integrated microtomography and optical imaging system with a rotating table that supports an imaging object, an optical stage, and separate optical and microtomography imaging systems. U.S. Pat. No. 9,770,220 (Stearns et al., Sep. 26, 2017, “Integrated Microtomography and Optical Imaging Systems”) discloses a rotating table that supports an imaging object, an optical stage, and separate optical and microtomography imaging systems. U.S. patent application 20170209083 (Zarandi et al., 2017, “Hand-Held Optical Scanner for Real-Time Imaging of Body Composition and Metabolism”) and U.S. patent Ser. No. 10/653,346 (Zarandi et al., May 19, 2020, “Hand-Held Optical Scanner for Real-Time Imaging of Body Composition and Metabolism”) disclose a handheld system for diffuse optical spectroscopic imaging of human tissue.

U.S. patent application 20060173352 (Lilge et al., 2006, “Optical Transillumination and Reflectance Spectroscopy to Quantify Disease Risk”) discloses a method of illuminating tissue of a mammal with light having wavelengths covering a pre-selected spectral range, detecting light transmitted through, or reflected from, the volume of selected tissue, and obtaining a spectrum of the detected light. U.S. patent application 20200116630 (Zhu, 2020, “Compact Guided Diffuse Optical Tomography System for Imaging a Lesion Region”) discloses a compact diffuse optical tomography system with laser diodes and a laser diode driver board. U.S. Pat. No. 5,876,339 (Lemire, Mar. 2, 1999, “Apparatus for Optical Breast Imaging”) discloses an optical breast imager with an adjustable volume which encloses a patient's breast.

U.S. Pat. No. 5,999,836 (Nelson et al., Dec. 7, 1999, “Enhanced High Resolution Breast Imaging Device and Method Utilizing Non-Ionizing Radiation of Narrow Spectral Bandwidth”) and U.S. Pat. No. 6,345,194 (Nelson et al., Feb. 5, 2002, “Enhanced High Resolution Breast Imaging Device and Method Utilizing Non-Ionizing Radiation of Narrow Spectral Bandwidth”) disclose breast imaging using collimated non-ionizing radiation in the near ultraviolet, visible, infrared, and microwave regions. U.S. Pat. No. 6,240,309 (Yamashita et al., May 29, 2001, “Optical Measurement Instrument for Living Body”), U.S. Pat. No. 6,640,133 (Yamashita et al., Oct. 28, 2003, “Optical Measurement Instrument for Living Body”), and U.S. Pat. No. 7,142,906 (Yamashita et al., Nov. 28, 2006, “Optical Measurement Instrument for Living Body”) disclose an optical measurement instrument which applies visible-infrared light to several positions on a patient.

U.S. patent application 20020045833 (Wake et al., Apr. 18, 2002, “Medical Optical Imaging Scanner Using Multiple Wavelength Simultaneous Data Acquisition for Breast Imaging”) discloses a scanner for a medical optical imaging device with an illumination source which directs emitted light into a breast positioned below a support surface. U.S. Pat. No. 6,571,116 (Wake et al., May 27, 2003, “Medical Optical Imaging Scanner Using Multiple Wavelength Simultaneous Data Acquisition for Breast Imaging”) and U.S. Pat. No. 6,738,658 (Wake et al., May 18, 2004, “Medical Optical Imaging Scanner Using Multiple Wavelength Simultaneous Data Acquisition for Breast Imaging”) disclose a medical optical imaging device with an illumination source that directs emitted light into a breast positioned below a support surface.

U.S. patent application publication 20040092826 (Corbeil et al., May 13, 2004, “Method and Apparatus for Optical Imaging”) and U.S. Pat. No. 7,809,422 (Corbeil et al., Oct. 5, 2010, “Method and Apparatus for Optical Imaging”) disclose a platform with a cavity into which one of the person's breasts is suspended for optical imaging. U.S. patent application publication 20070287897 (Faris, Dec. 13, 2007, “Optical Vascular Function Imaging System and Method for Detection and Diagnosis of Cancerous Tumors”) discloses an in-vivo optical imaging system and method of identifying unusual vasculature associated with tumors. U.S. Pat. No. 8,027,711 (Jones et al., Sep. 27, 2011, “Laser Imaging Apparatus with Variable Patient Positioning”) discloses a tabletop to support a patient in front-down position and an opening to permit a breast of the patient to be vertically pendant below the tabletop.

U.S. Pat. No. 8,224,426 (Lilge et al., Jul. 17, 2012, “Optical Transillumination and Reflectance Spectroscopy to Quantify Disease Risk”) discloses spectroscopic tissue volume measurements with non-ionizing radiation to detect pre-disease transformations in tissue. U.S. patent application publication 20160066811 (Mohamadi, Mar. 10, 2016, “Handheld and Portable Scanners for Millimeter Wave Mammography and Instant Mammography Imaging”) discloses an array of ultra-wide band radio frequency sensors for breast imaging. U.S. Pat. No. 9,513,276 (Tearney et al., Dec. 6, 2016, “Method and Apparatus for Optical Imaging via Spectral Encoding”) disclose a method, apparatus and arrangement for obtaining information associated with a sample such as a portion of an anatomical structure. U.S. patent application publication 20170007187 (Breneisen et al., Jan. 12, 2017, “Cancer Detector Using Deep Optical Scanning”) discloses Deep Optical Scanning (DEOS) for the detection of breast cancer and the determination of response to therapy.

U.S. Pat. No. 9,597,046 (Goossen et al., Mar. 21, 2017, “Method and Device for Imaging Soft Body Tissue Using X-Ray Projection and Optical Tomography”) discloses breast imaging using X-ray projection techniques and optical tomography techniques. U.S. patent application 20170105625 (Eum, Apr. 20, 2017, “Diagnostic Device of Optics Type for Breast”) discloses an optical breast diagnostic apparatus with a hemispherical cover. U.S. patent Ser. No. 10/200,655 (Kim et al., Feb. 5, 2019, “Tomographic Imaging Methods, Devices, and Systems”) discloses a multispectral bioluminescence optical tomography algorithm makes use of a partial differential equation (PDE) constrained approach. U.S. patent Ser. No. 10/215,636 (Fujii et al., Feb. 26, 2019, “Imaging Device Provided With Light Source That Emits Pulsed Light and Image Sensor”) discloses an imaging device with a light source that emits pulsed light at different wavelengths. U.S. patent Ser. No. 10/506,181 (Delgado et al., Dec. 10, 2019, “Device for Optical Imaging”) discloses the capture of an infrared image.

Turning to the non-patent literature, Ahmed et al., (2021), “Differential Optical Absorption Spectroscopy-Based Refractive Index Sensor for Cancer Cell Detection,” Optical Review, 28, 134-143, discloses a spectroscopic optical sensor for cancerous cell detection in various parts of the human body. Altoe et al., (2019), “Diffuse Optical Tomography of the Breast: A Potential Modifiable Biomarker of Breast Cancer Risk with Neoadjuvant Chemotherapy,” Biomedical Optics Express, Aug. 1, 2019, 10(8), 4305-4315, studied whether a diffuse optical tomography breast imaging system (DOTBIS) can provide a comparable optical-based image index of mammographic breast density. Altoe et al., (2021), “Changes in Diffuse Optical Tomography Images During Early Stages of Neoadjuvant Chemotherapy Correlate with Tumor Response in Different Breast Cancer Subtypes”, Clinical Cancer Research, Apr. 1, 2021, 27(7), 1949-1957, studied changes in optically derived parameters acquired with a diffuse optical tomography breast imaging system (DOTBIS) in the tumor volume of patients with breast carcinoma receiving neoadjuvant chemotherapy (NAC).

Altoe et al., (2021), “Effects of Neoadjuvant Chemotherapy on the Contralateral Non-Tumor-Bearing Breast Assessed by Diffuse Optical Tomography,” Breast Cancer Research, 2021, 23, 16, studied whether changes in optically derived parameters acquired with a diffuse optical tomography breast imager system (DOTBIS) in the contralateral non-tumor-bearing breast in patients administered neoadjuvant chemotherapy (NAC) for breast cancer are associated with pathologic complete response (pCR). Anabestani et al. (2022), “Advances in Flexible Organic Photodetectors: Materials and Applications,” Nanomaterials, 2022, 12(21), 3775, discusses recent advances in flexible organic photodetectors, including their applications in health-monitoring, X-ray detection, and imaging. Anderson et al., (2017), “Optical Mammography in Patients with Breast Cancer Undergoing Neoadjuvant Chemotherapy: Individual Clinical Response Index,” Academic Radiology, October, 2017, 24(10), 1240-1255, discloses an optical mammography study to develop quantitative measures of pathologic response to neoadjuvant chemotherapy (NAC) in patients with breast cancer.

Angelo et al., (2018), “Review of Structured Light in Diffuse Optical Imaging,” Journal of Biomedical Optics, Sep. 14, 2018, 24(7), 071602, discloses diffuse optical imaging probes in living tissue enabling structural, functional, metabolic, and molecular imaging. Applegate et al., (2018), “Multi-Distance Diffuse Optical Spectroscopy with a Single Optode via Hypotrochoidal Scanning,” Optics Letters, 2018, 43, 747-750, studied a new method of frequency-domain diffuse optical spectroscopy (FD-DOS) to rapidly acquire a wide range of source-detector (SD) separations by mechanically scanning a single SD pair. Applegate et al. (2020), “Recent Advances in High Speed Diffuse Optical Imaging in Biomedicine,” APL Photonics, 2020, 5(4), 040802, 21, reviews recent advances in acquisition and processing speed for several Diffuse Optical Imaging modalities.

Chae et al., (2020), “Development of Digital Breast Tomosynthesis and Diffuse Optical Tomography Fusion Imaging for Breast Cancer Detection,” Scientific Reports, 10, 13127 (2020), studied a new digital breast tomosynthesis (DBT)/DOT fusion imaging technique for breast cancer detection. Chitnis et al. (2016), “Towards a Wearable Near Infrared Spectroscopic Probe for Monitoring Concentrations of Multiple Chromophores in Biological Tissue In Vivo,” Review of Scientific Instruments, June, 2016, 87(6), 065112, discloses a wearable multi-wavelength technology for functional near-infrared spectroscopy with an 8-wavelength light emitting diode (LED) source. Cinquino et al. (2021), “Light-Emitting Textiles: Device Architectures, Working Principles, and Applications,” Micromachines, (Special Issue Emerging and Disruptive Next-Generation Technologies for POC: Sensors, Chemistry and Microfluidics for Diagnostics), 2021, 12(6), 652, discusses applications of light-emitting fabrics, including Organic LEDs.

Cochran et al., (2019), “Hybrid Time-Domain and Continuous-Wave Diffuse Optical Tomography Instrument with Concurrent, Clinical Magnetic Resonance Imaging for Breast Cancer Imaging,” Journal of Biomedical Optics, January, 2019, 24(5), 1-11, discusses diffuse optical tomography (DOT) for three-dimensional (3-D) maps of tissue optical and physiological properties in human tissue. Costa et al. (2019), “Flexible Sensors: From Materials to Applications,” Technologies (Special Issue, Reviews and Advances in Internet of Things Technologies), 2019, 7(2), 35, reviews the current state of flexible sensor technologies and the impact of material developments on this field. Durduran et al., (2010, 2010), “Diffuse Optics for Tissue Monitoring and Tomography,” Reports on Progress in Physics, 2010, 73(7), 076701, discloses using near-infrared or diffuse optical spectroscopy to measure tissue hemodynamics.

Fakayode et al., (2020), “Molecular (Raman, NIR, and FTIR) Spectroscopy and Multivariate Analysis in Consumable Products Analysis,” Applied Spectroscopy, reviews, 55:8, 647-723, reviews the use of Raman, near-infrared (NIR), and Fourier-transform infrared (FTIR) spectrometers to evaluate consumable products such as food. Fantini et al., (2001), “Optical Spectroscopy and Imaging of Tissues,” NSF Award #0093840, Jun. 1, 2001, studied development of new improved methods and instrumentation for biomedical applications of near-infrared spectroscopy and imaging. Fantini (2005), “Optical Spectroscopy and Imaging of Tissues”, NSF Award, 2005 (abstract only viewed), researched techniques for optical spectroscopy and imaging of biological tissues. Fantini et al., (2012), “Near-Infrared Optical Mammography for Breast Cancer Detection with Intrinsic Contrast,” Annals of Biomedical Engineering, February, 2012, 40(2), 398-407, reviews optical methods to detect breast cancer on the basis of increased opacity.

Farmani et al., (2020), “Optical Nanosensors for Cancer and Virus Detections,” Micro and Nano Technologies, Nanosensors for Smart Cities, Chapter 25, Han et al. editors, Elsevier, 2020, 419-432, ISBN 9780128198704, discusses photonic crystal (PhC)-based optical nanosensors. Feng et al. (2021), “MRI Guided Wearable Near Infrared Spectral Tomography: Simulation Study,” Proceedings SPIE, 11639, Optical Tomography and Spectroscopy of Tissue XIV, 116390D, Mar. 5, 2021, discloses a new low-cost imaging system for MRI-guided Near-Infrared Spectral Tomography (MRI-NIRST) for breast cancer detection. Flexman et al., (2008), “The Design and Characterization of a Digital Optical Breast Cancer Imaging System,” 30th Annual International Conference of the IEEE Engineering in Medicine and Biology Society, 2008, 3735-3738, discusses how optical imaging has the potential to play a major role in breast cancer screening and diagnosis due to its ability to image cancer characteristics such as angiogenesis and hypoxia.

Ghijsen et al., (2018), “Quantitative Real-Time Optical Imaging of the Tissue Metabolic Rate of Oxygen Consumption,” Journal of Biomedical Optics, Mar. 24, 2018, 23(3), 036013, discloses a noncontact method for quantitatively mapping tMRO2 over a wide, scalable field of view. Grosenick et al., (2016), “Review of Optical Breast Imaging and Spectroscopy,” Journal of Biomedical Optics, September, 2016, 21(9), 091311, reviews the monitoring neoadjuvant chemotherapy and breast cancer risk assessment via optical breast imaging and spectroscopy. Gunther et al., (2018), “Dynamic Diffuse Optical Tomography for Monitoring Neoadjuvant Chemotherapy in Patients with Breast Cancer,” Radiology, June, 2018; 287(3): 778-786, identifies dynamic optical imaging features associated with pathologic response in patients with breast cancer during neoadjuvant chemotherapy.

Hoi et al., (2018), “Non-Contact Dynamic Diffuse Optical Tomography Imaging System for Evaluating Lower Extremity Vasculature,” Biomedical Optics Express, 2018, 9, 5597-5614, discloses a multi-view non-contact dynamic diffuse optical tomographic imaging system for the clinical evaluation of vasculature in the lower extremities. Imamura et al., (2018), “In Vivo Optical Imaging of Cancer Cell Function and Tumor Microenvironment,” Cancer Science, 2018, 109, 912-918, discusses in vivo optical imaging using fluorescence and bioluminescence. Intes et al. (2004), “Time-Domain Optical Mammography Softscan: Initial Results On Detection and Characterization of Breast Tumors,” Proceedings SPIE 5578, Photonics North 2004: Photonic Applications in Astronomy, Biomedicine, Imaging, Materials Processing, and Education, Dec. 9, 2004 presents initial results obtained using a breast-imaging system developed by Advanced Research Technologies comprising a 4-wavelength time-resolved scanning system.

Jeong et al., (2020), “Emerging Advanced Metasurfaces: Alternatives to Conventional Bulk Optical Devices,” Microelectronic Engineering, 2020, Vol. 220, 111146, ISSN 0167-9317, discusses the use of optical metasurfaces as color filters, metalenses, beam generators or splitters, and meta-holograms. Jiang et al. (2021), “MRI-Guide Near Infrared Spectroscopic Tomographic Imaging System with Wearable Optical Breast Interface for Breast Imaging,” Proceedings SPIE, 11639, Optical Tomography and Spectroscopy of Tissue XIV, 116391J, 3/5/2021, discloses a new photo-detector (PD) and source fiber based wearable MRI-guide near infrared spectroscopic tomographic imaging (MRg-NIRST) system. Joshi et al., (2018), “Targeted Optical Imaging Agents in Cancer: Focus on Clinical Applications,” Contrast Media and Molecular Imaging, Aug. 27, 2018, discusses molecular imaging for in vivo visualization of cancer over time based on biological mechanisms of disease activity.

Jung et al. (2015), “Non-Contact Deep Tissue Imaging using a Hand-Held Near-infrared Optical Scanner,” Journal of Medical Diagnostic Methods, Mar. 24, 2015, 4(2), 1-10, discloses fiber-free non-contact near-infrared (NIR) imaging devices using wide-field detectors Khan (2013), “Image Reconstruction in Diffuse Optical Tomography With Sparsity Constraints”, NSF Award, 2013 (abstract only viewed), researched the use of sparsity-constrained regularization for solving the diffuse optical tomography inverse problem. Kim et al., (2016), “US-Localized Diffuse Optical Tomography in Breast Cancer: Comparison with Pharmacokinetic Parameters of DCE-MRI and With Pathologic Biomarkers,” BMC Cancer, Feb. 1, 2016, 16:50, discloses correlating parameters of ultrasonography-guided diffuse optical tomography with the pharmacokinetic features of dynamic contrast-enhanced MRI and pathologic markers of breast cancer. Koetse et al., (2007), “Optical Sensor Array Platform Based on Polymer Electronic Devices,” Proceedings SPIE 6739, Electro-Optical Remote Sensing, Detection, and Photonic Technologies and Their Applications, 67391D, Nov. 7, 2007, discusses devices based on polymer semiconductors fabricated with thin film technology.

Koomson, 2019), “A Noninvasive Biological Research Tool for Measurement of Tissue and Cerebral Oxygenation,” NSF Award #1919038, Jul. 15, 2019, (abstract only viewed) investigates compact wearable devices with advanced NIRS capability. Krishnamurthy, 2018), “Using Near-Infrared Spectroscopy to Study Static and Dynamic Hemoglobin Contrast Associated with Breast Cancer,” Tufts University, Dissertation, 2018, discloses an instrument for diffuse optical mammography with parallel plate geometry. Leo et al. (2017), “Optical Imaging of the Breast: Basic Principles and Clinical Applications,” American Journal of Roentgenology, 2017, 209:1, 230-238, discloses summarizes the physical principles, technology features, and first clinical applications of optical imaging techniques to the breast. Li et al. (2018), “Sensitive and Wearable Optical Microfiber Sensor for Human Health Monitoring,” Advanced Materials Technologies, 2018, 3, 1800296, discloses a sensor with a hybrid plasmonic microfiber knot resonator embedded in a polydimethylsiloxane membrane.

Liu et al., (2018), “Diffuse Optical Spectroscopy for Monitoring the Responses of Patients with Breast Cancer to Neoadjuvant Chemotherapy: A Meta-Analysis,” Medicine, 2018, 97(41), 12683, investigated the potential of diffuse optical spectroscopy (DOT) for monitoring the responses of patients with breast cancer to neoadjuvant chemotherapy (NAC). Liu et al., (2020), “Recent Progress in Flexible Wearable Sensors for Vital Sign Monitoring,” Sensors, 2020, 20(14), 4009, discusses the development of flexible electronic materials, as well as the wide development and application of smartphones, the cloud, and wireless systems, flexible wearable sensor technology. Liu et al., (2021), “Simultaneous Measurements of Tissue Blood Flow and Oxygenation Using a Wearable Fiber-Free Optical Sensor,” Journal of Biomedical Optics, Jan. 29, 2021, 26(1), 012705, discusses a wearable dual-wavelength diffuse speckle contrast flow oximetry (DSCFO) device for simultaneous measurements of blood flow and oxygenation variation in deep tissues.

Lutzweiler et al., (2013), “Optoacoustic Imaging and Tomography: Reconstruction Approaches and Outstanding Challenges in Image Performance and Quantification,” Sensors, 2013, 13(3), 7345-7384, reviews optoacoustic imaging from image reconstruction and quantification perspectives. Ma et al. (2020b), “Fiber-Free Parallel-Plane Continuous Wave Breast Diffuse Optical Tomography System,” SPIE 11229, Advanced Biomedical and Clinical Diagnostic and Surgical Guidance Systems XVIII, Proceedings, 112290L, Feb. 21, 2020 discusses near infrared diffuse optical tomography (DOT) for detecting breast cancer. Mabou et al., (2018), “Breast Cancer Detection Using Infrared Thermal Imaging and a Deep Learning Model,” Sensors, 2018, 18(9), 2799, discloses the use of infrared digital imaging for breast cancer detection based on thermal comparison between a healthy breast and a breast with cancer.

Moreno et al. (2019), “Evaluation on Phantoms of the Feasibility of a Smart Bra to Detect Breast Cancer in Young Adults,” Sensors, 2019, 19(24), 5491, discloses the use of breast tissue phantoms to investigate the feasibility of quantifying breast density and detecting breast cancer tumors using a smart bra. Nguyen et al., (2020), “Preliminary Development of Optical Computed Tomography (Optical CT) Scanner Using Transillumination Imaging NAD,” Conference: International Symposium on Applied Science 2019, Hochiminh City, Vietnam, May 14, 2020, discusses the use of near-infrared transillumination imaging for biomedical applications such as human biometrics and animal experiments. Pan et al., (2020), “A Multifunctional Skin-Like Wearable Optical Sensor Based on an Optical Micro-/Nanofibre,” Nanoscale, 2020, Issue 33, discusses multifunctional skin-like sensors for next-generation healthcare, robotics, and bioelectronics.

Park et al., (2013), “Multispectral Imaging Using Polydimethylsiloxane (PDMS) Embedded Vertical Silicon Nanowires,” OSA Technical Digest (online) (Optical Society of America, 2013), paper CTu3O.1, reports on the demonstration of a compact multispectral imaging system that uses vertical silicon nanowires for a filter array. Park et al., (2015), “Vertically Stacked Photodetector Devices Containing Silicon Nanowires with Engineered Absorption Spectra,” ACS Photonics, Mar. 16, 2015, 2(4), 544-549, discloses a vertically stacked photodetector device containing silicon nanowire photodetectors formed above a silicon substrate that also contains a photodetector. Perumal et al., (2019), “Near Infra-Red Polymeric Nanoparticle Based Optical Imaging in Cancer Diagnosis,” Journal of Photochemistry and Photobiology, Biology, 2019, Vol. 199, 111630, ISSN 1011-1344, reviews the recent progress in NIRF polymeric nanoparticles used for optical imaging particularly on cancer diagnosis.

Pinti et al. (2018), “A Review on the Use of Wearable Functional Near-Infrared Spectroscopy in Naturalistic Environments,” Japanese Psychology Research, October 2018, 60(4), 347-373, reviews the use of wearable fNIRS in naturalistic settings in the field of cognitive neuroscience. Qiu (2018), “Implantable Ultra-low Power VO2 MEMS Scanner Based Surface-Enhanced Raman Spectroscope for Wide-field Tumor Imaging in Free Moving Small Animals”, NSF Award, 2018 (abstract only viewed), discloses tumor-targeting surface enhanced Raman scattering nanoparticles based on multiplexed Raman spectroscopy. Rahman et al., (2016), “Electromagnetic Performances Analysis of an Ultra-Wideband and Flexible Material Antenna in Microwave Breast Imaging: To Implement a Wearable Medical Bra,” Scientific Reports, 2016, Vol. 6, 38906, discloses a compact and ultra-wide band antenna on a flexible substrate for microwave imaging.

Ray et al. (2017), “A Systematic Review of Wearable Systems for Cancer Detection: Current State and Challenges,” Journal of Medical Systems, Oct. 2, 2017, 41(11), 180, reviews cancer detection using wearable systems, including sensor-based smart systems with a microcontroller, Bluetooth module, and smart phone. Robbins et al. (2021), “Two-Layer Spatial Frequency Domain Imaging of Compression-Induced Hemodynamic Changes in Breast Tissue,” Journal of Biomedical Optics, 5/24/2021, 26(5), 056005, studied hemodynamic changes in response to localized breast compression using a handheld SFDI device. Roblyer et al. (2020b), “Tracking Breast Cancer Therapies with Handheld and Wearable Diffuse Optics,” Biophotonics Congress: Biomedical Optics 2020 (Translational, Microscopy, OCT, OTS, BRAIN), OSA Technical Digest (Optical Society of America, 2020), paper TM4B.1 disclose an NIR-II imaging system), “Detection of Optically Luminescent Probes using Hyperspectral and Diffuse Imaging in Near-infrared” (DOLPHIN) for noninvasive real-time tracking of a 0.1 mm-sized fluorophore through the gastrointestinal tract of a mouse.

Saikia et al. (2017), “A Cost-Effective LED and Photodetector Based Fast Direct 3D Diffuse Optical Imaging System,” Proc. SPIE 10412, Diffuse Optical Spectroscopy and Imaging VI, Jul. 28, 2017, European Conferences on Biomedical Optics, 2017, Munich, Germany, discloses a cost-effective and high-speed 3D diffuse optical tomography system using high power LED light sources and silicon photodetectors. Saikia et al. (2019), “A Point-of-Care Handheld Region-of-Interest (ROI) 3D Functional Diffuse Optical Tomography (fDOT) System,” Proc. SPIE 10874, Optical Tomography and Spectroscopy of Tissue XIII, Mar. 1, 2019, discloses a 3D Functional Diffuse Optical Tomography (fDOT) system based on an Internet-of-things (IoT) concept. Satharasinghe et al. (2018), “Photodiodes Embedded Within Electronic Textiles,” Science Reports, 2018, 8, 16205, discloses a novel photodiode-embedded yarn with possible applications including monitoring body vital signs.

Schoustra et al. (2021), “Pendant Breast Immobilization and Positioning in Photoacoustic Tomographic Imaging,” Photoacoustics, 2021, 21, 100238 describes the design, development and added value of breast-supporting cups to immobilize and position the pendant breast in photoacoustic tomographic imaging. Shokoufi et al. (2017), “Novel Handheld Diffuse Optical Spectroscopy Probe for Breast Cancer Assessment: Clinical Study,” Journal of Biomedical Science, 6(5), 34, discloses a hand-held continuous-wave radio-frequency modulated diffuse optical spectroscopy probe. Soliman et al., (2010), “Functional Imaging Using Diffuse Optical Spectroscopy of Neoadjuvant Chemotherapy Response in Women with Locally Advanced Breast Cancer,” Clinical Cancer Research, Apr. 20, 2010, 15, 2605-2614, discloses functional imaging with tomographic near-infrared diffuse optical spectroscopy to measure tissue concentration of deoxyhemoglobin, oxyhemoglobin, percent water, and scattering power.

Spink et al. (2020), “High Optode-Density Wearable Probe for Monitoring Breast Tumor Dynamics During Neoadjuvant Chemotherapy,” Biophotonics Congress: Biomedical Optics 2020 (Translational, Microscopy, OCT, OTS, BRAIN), OSA Technical Digest (Optical Society of America, 2020), paper TTu1B.2 disclose an NIR-II imaging system), “Detection of Optically Luminescent Probes using Hyperspectral and diffuse Imaging in Near-infrared” (DOLPHIN). Spink et al. (2021), “High Optode-Density Wearable Diffuse Optical Probe for Monitoring Paced Breathing Hemodynamics in Breast Tissue,” Journal of Biomedical Optics, Jun. 2, 2021, 26(6), 062708, discloses a high optode-density wearable continuous wave diffuse optical probe for the monitoring of breathing hemodynamics in breast tissue. Tank et al. (2020), “Diffuse Optical Spectroscopic Imaging Reveals Distinct Early Breast Tumor Hemodynamic Responses to Metronomic and Maximum Tolerated Dose Regimens,” Breast Cancer Research, 2020, 22, 29 reports on a dual-center study which examined 54 breast tumors receiving NAC measured with DOSI before therapy and the first week following chemotherapy administration.

Teng (2018), “A Wearable Near-Infrared Diffuse Optical System for Monitoring in Vivo Breast Tumor Hemodynamics During Chemotherapy Infusions,” Boston University, Dissertation, 2018, discloses a new wearable diffuse optical device to investigate if very early timepoints during a patient's first chemotherapy infusion are predictive of overall response (pCR versus non-pCR) to NAC. Teng et al. (2017), “Wearable Near-Infrared Optical Probe for Continuous Monitoring During Breast Cancer Neoadjuvant Chemotherapy Infusions,” Journal of Biomedical Optics, 22(1), 14001 presents a new continuous-wave wearable diffuse optical probe for investigating the hemodynamic response of locally advanced breast cancer patients during neoadjuvant chemotherapy infusions. Tiwari et al. (2022), “Role of Sensor Technology in Detection of the Breast Cancer,” BioNanoScience, 2022, 12, 639-659, reviews different sensors developed to detect breast cancer over the past few years.

Tromberg et al., (2016), “ACRIN 6691 Investigators. Predicting Responses to Neoadjuvant Chemotherapy in Breast Cancer,” Cancer Research, Aug. 15, 2016, 76(20), 5933-5944, investigates whether changes from baseline to mid-therapy in a diffuse optical spectroscopic imaging (DOSI)-derived imaging endpoint, the tissue optical index, predict pathologic complete response in women undergoing breast cancer neoadjuvant chemotherapy. Uddin et al., (2020a), “Optimal Breast Cancer Diagnostic Strategy Using Combined Ultrasound and Diffuse Optical Tomography,” Biomedical Optics Express, 11(5), 2722-2737, presents a two-stage diagnostic strategy that is both computationally efficient and accurate. Upputuri, (2019), “Photoacoustic Imaging in the Second Near-Infrared Window: A Review,” Journal of Biomedical Optics, Apr. 9, 2019, 24(4), 040901, discusses photoacoustic (PA) imaging that combines optical excitation and ultrasound detection.

Vavadi et al., (2018), “Compact Ultrasound-Guided Diffuse Optical Tomography System for Breast Cancer Imaging,” Journal of Biomedical Optics, 2018, 24(2), 1-9, discusses an ultrasound-guided DOT system. Wang et al. (2020), “Development of a Prototype of a Wearable Flexible Electro-Optical Imaging System for the Breast,” Biophotonics Congress: Biomedical Optics 2020 (Translational, Microscopy, OCT, OTS, BRAIN), OSA Technical Digest (Optical Society of America, 2020), paper TM4B.4, discloses a wearable breast imaging system which combines a garment and a flexible electronic system. Yu et al. (2010), “Near-Infrared, Broad-Band Spectral Imaging of the Human Breast for Quantitative Oximetry: Applications to Healthy and Cancerous Breasts,” Journal of Innovative Optical Health Sciences, October 2010, 03(4):267-277 discusses the examination of ten human subjects with a previously developed instrument for near-infrared diffuse spectral imaging of the female breast.

Yuan et al. (2014), “Light-Emitting Diode-Based Multiwavelength Diffuse Optical Tomography System Guided by Ultrasound,” Journal of Biomedical Optics, Dec. 4, 2014, 19(12) 126003, discloses a low-cost DOT system using LEDs of four wavelengths in the NIR spectrum as light sources. Zhang et al., (2020), “Efficacy of Shear-Wave Elastography Versus Dynamic Optical Breast Imaging for Predicting the Pathological Response to Neoadjuvant Chemotherapy in Breast Cancer,” European Journal of Radiology, 2020, 129, 109098, discusses the value of shear-wave elastography (SWE) parameters and dynamic optical breast imaging features for predicting pathological responses to neoadjuvant chemotherapy (NACT) in breast cancer (BC).

Zhao et al. (2021), “High Resolution, Deep Imaging Using Confocal Time-of-Flight Diffuse Optical Tomography,” IEEE Transactions on Pattern Analysis and Machine Intelligence, Jul. 1, 2021, 43(7), 2206-2219, discloses how time-of-flight diffuse optical tomography (ToF-DOT) can achieve millimeter spatial resolution in the highly scattered diffusion regime. Zhao et al. (2022), “MRI-Guided Near-Infrared Spectroscopic Tomography (MRg-NIRST): System Development for Wearable, Simultaneous NIRS and MRI Imaging,” Proc. SPIE 11952, Multimodal Biomedical Imaging XVII, Mar. 2, 2022, discloses a novel wearable MRg-NIRST system for breast cancer detection with eight flex circuit strips, each with six photodetectors (PDs) and six source fibers.

Zhu et al. (2020), “A Review of Optical Breast Imaging: Multi-Modality Systems for Breast Cancer Diagnosis,” European Journal of Radiology, August 2020, 129:109067, reviews optical breast imaging using multi-modality platforms. Zhu et al., (2021), “Early Assessment Window for Predicting Breast Cancer Neoadjuvant Therapy Using Biomarkers, Ultrasound, and Diffuse Optical Tomography,” Breast Cancer Research and Treatment, 2021, assesses the utility of tumor biomarkers, ultrasound (US) and US-guided diffuse optical tomography (DOT) in early prediction of breast cancer response to neoadjuvant therapy (NAT).

### SUMMARY OF THE INVENTION

This invention is a multi-layer wearable device for optical detection of breast cancer. It can be embodied as a smart bra or as a bra insert which is inserted between a conventional bra and a person's breast. This device has four layers: an air-gap-reducing layer which is closest to the breast; an optical layer with a plurality of light emitters and light detectors; an expandable layer with a plurality of expandable components; and an outer structural layer. Light from the light emitters which has been transmitted through and/or reflected from breast tissue and received by the light detectors is analyzed to detect and/or image abnormal breast tissue.

This device addresses two key limitations of devices in the prior art for optical detection of breast cancer. First, the air-gap-reducing layer of this device reduces errors due to air gaps between optical components and the surface of the breast. Second, the expandable layer of this device reduces light scattering through the breast by gently compressing the breast.

### INTRODUCTION TO THE FIGURES

FIGS. 1 and 2 show two views of a multi-layer wearable device for optical detection of breast cancer. FIG. 1 shows this device in an unexpanded configuration. FIG. 2 shows this device in an expanded configuration.

### DETAILED DESCRIPTION OF THE FIGURES

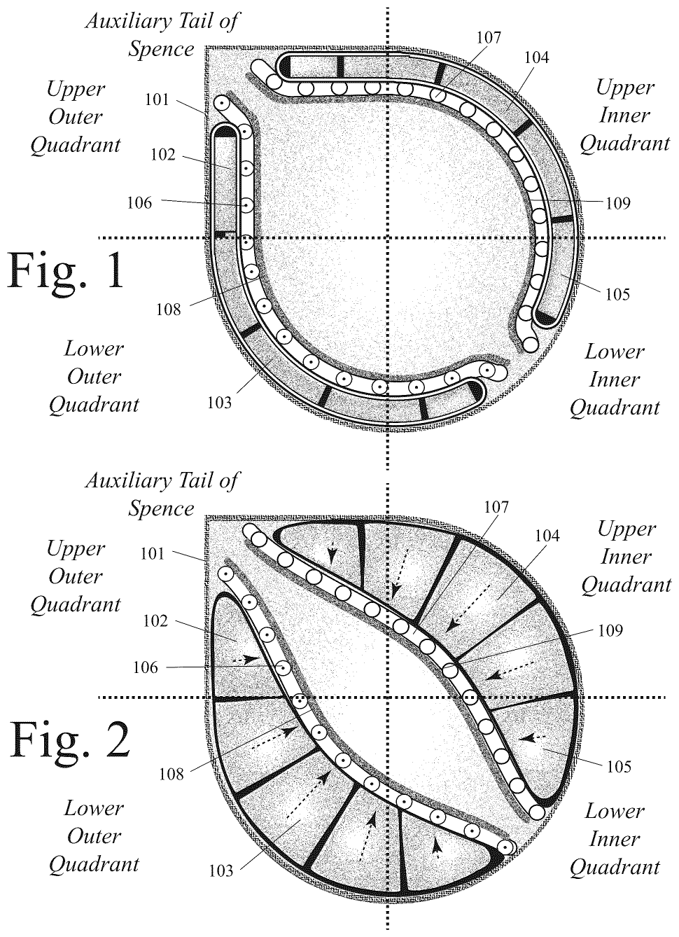

FIGS. 1 and 2 show two sequential views of a multi-layer device for optical detection of breast cancer which is configured to be worn on a person's breast comprising: (a) an air-gap-reducing layer which is configured to be worn on the surface of a person's breast, wherein the air-gap-reducing layer is transparent or has optical characteristics like those of breast tissue, and wherein a first part (e.g. portion, part, section, and/or half) of the air-gap-reducing layer is on a first side of a virtual plane and a second part of the air-gap-reducing layer is on a second (e.g. opposite) side of the virtual plane; (b) an optical layer with a plurality of light emitters and light detectors, wherein a first part of the optical layer is on a first side of the virtual plane and a second part of the optical layer is on a second side of the virtual plane; (c) an expandable layer with a plurality of expandable components, wherein a first part of the expandable layer is on a first side of the virtual plane and a second part of the expandable layer is on a second side of the virtual plane; and (d) a structural layer which reduces expansion of the expandable components away from the breast; wherein the optical layer is between the air-gap-reducing layer and the expandable layer, wherein the expandable layer is between the optical layer and the structural layer, wherein the device has an unexpanded configuration in which the expandable components are not expanded, wherein the device has an expanded configuration in which the expandable components are expanded, wherein there is a first average distance between the first part of the optical layer and the second part of the optical layer when the device is in the unexpanded configuration, wherein there is a second average distance between the first part of the optical layer and the second part of the optical layer when the device is in the expanded configuration, and wherein the second average distance is less than the first average distance.

With respect to specific components, FIGS. 1 and 2 show a multi-layer device for optical detection of breast cancer which is configured to be worn on a person's breast comprising: (a) an air-gap-reducing layer which is configured to be worn on the surface of a person's breast, wherein the air-gap-reducing layer is transparent or has optical characteristics like those of breast tissue, and wherein a first part (e.g. portion, part, section, and/or half) 108 of the air-gap-reducing layer is on a first side of a virtual plane and a second part 109 of the air-gap-reducing layer is on a second (e.g. opposite) side of the virtual plane; (b) an optical layer with a plurality of light emitters (including 106) and light detectors (including 107), wherein a first part of the optical layer is on a first side of the virtual plane and a second part of the optical layer is on a second side of the virtual plane; (c) an expandable layer with a plurality of expandable components (including 102, 103, 104, and 105), wherein a first part of the expandable layer is on a first side of the virtual plane and a second part of the expandable layer is on a second side of the virtual plane; and (d) a structural layer 101 which reduces expansion of the expandable components away from the breast; wherein the optical layer is between the air-gap-reducing layer and the expandable layer, wherein the expandable layer is between the optical layer and the structural layer, wherein the device has an unexpanded configuration in which the expandable components are not expanded, wherein the device has an expanded configuration in which the expandable components are expanded, wherein there is a first average distance between the first part of the optical layer and the second part of the optical layer when the device is in the unexpanded configuration, wherein there is a second average distance between the first part of the optical layer and the second part of the optical layer when the device is in the expanded configuration, and wherein the second average distance is less than the first average distance.

FIG. 1 shows this device at a first time when the device is in its unexpanded configuration, wherein expandable components 102, 103, 104, and 105 have not yet been expanded. FIG. 2 shows this device at a second time when the device is in its expanded configuration, wherein expandable components 102, 103, 104, and 105 have expanded to gently compress the breast for improved optical scanning Gentile compression of the breast reduces the maximum thickness of the breast for better light transmission and also reduces air gaps between optical components and the surface of the breast.

FIGS. 1 and 2 also illustrate the four quadrants of the breast: upper outer quadrant, upper inner quadrant, lower inner quadrant, and lower outer quadrant. Divisions between these quadrants are shown in FIGS. 1 and 2 by vertical and horizontal dotted lines. Illustration of these four quadrants provides a useful framework for referencing specific areas of the breast when specifying design variations for this device. FIGS. 1 and 2 also show the Auxiliary Tail of Spence, wherein the upper outer quadrant of the breast attaches to the chest wall. Showing the Auxiliary Tail of Spence is useful because the upper outer quadrant and Auxiliary Tail of Spence are more likely to develop abnormal tissue than other areas of the breast. In this example, the device has a teardrop-shaped perimeter which spans the four quadrants of the breast and also the Auxiliary Tail of Spence.

In an example, this device can be embodied as a smart bra. In an example, this device can be embodied as a cup of a smart bra. In an example, right-side and left-side versions of this device can be embodied as right-side and left-side cups of a smart bra. In an example, this device can further comprise other components selected from the group consisting of: power source, data processor, wireless data transmitter, wireless data receiver, air pump, and liquid pump. In a smart bra embodiment, these other components can be located on the back (e.g. strap) portion of the bra. In an example, a pump can be separate, removably-connected to the bra for expansion of the expandable components, and detached for wearing the bra.

In an example, a device for breast tissue imaging and/or identifying abnormal tissue in a breast can be embodied in a wearable garment (e.g. “smart bra”) with a plurality of light emitters and light detectors. In an example, this device can be embodied in a smart bra which includes optical sensors and electronic components. In an example, expandable components, light emitters, and light detectors can be located within the concavity of a cup in a smart bra. In an example, this device can be embodied in smart bras which come in different sizes corresponding to conventional smart bra sizes. In an example, this device can be embodied in a smart bra which can be custom fitted to a particular breast size and shape by selective expansion of expandable components in an expandable layer in a bra cup.

In an example, a cup on one side (e.g. the right side or left side) of a smart bra can have a similar (e.g. same, but symmetric) configuration of expanding components, light emitters, and light detectors as a cup on the other side (e.g. the left side or right side) of the smart bra. In an example, a cup on one side of a smart bra can have a similar (e.g. same, but reflected across a central vertical plane) configuration of expanding components, light emitters, and light detectors as a cup on the other side of the smart bra.

In an example, a device can be embodied in a smart bra wherein some portions of a cup are reinforced by wires. In an example, an outer structural layer of a cup can be reinforced with wires so that pressure from expansion of an inner expandable layer is directed primarily inward toward a breast to compress the breast for better optical scanning. In an example, some portions of a cup (e.g. of a structural layer of the cup) can be selectively reinforced with wires while other portions of the cup are not. In an example, portions of a cup (e.g. of a structural layer of the cup) which are farther from a virtual plane can be reinforced with wire, but portions of the cup which are closer to virtual plane are not. In an example, this virtual plane can be an oblique plane which spans from the upper outer quadrant (and the Auxiliary Tail of Spence) to the lower inner quadrant. Selectively placement of reinforcing wire in a cup can help to flatten the breast along the virtual plane to improve optical scanning.

In an example, a device can be bra insert. In an example, a device can be removably-attached to the concave interior of a bra cup (e.g. by hook-and-loop material) so that it is held in place for optical scanning, but can be removed for washing the bra without exposing optical sensors (or other electronics) to water and soap. In an example, a device can be removably-attached to the concave interior of a bra cup by an attachment mechanism (e.g. hook-and-loop material, snap, clip, or magnet) so that it is held in place for optical scanning, but can also be removed for washing the bra without exposing optical sensors (or other electronics) to water and soap.

In an example, a device can be embodied in a smart bra with a right-side cup and a left-side cup, each having optical sensors. In an example, a device can be embodied in a smart bra with a right-side cup and a left-side cup, each having light emitters and light detectors. In an example, the size of an interior concavity of a smart bra cup can correspond to the cup size of a conventional smart bra, but the exterior size of the smart bra cup can be larger due to space occupied by expandable components, light emitters, and light detectors. In an example, data from light detectors in a smart bra can be analyzed to identify and/or image abnormal breast tissue. In an example, this analysis can be done in a local data processor which is part of the device. In an example, data from light detectors can be wirelessly transmitted to a separate and/or remote data processor to identify and/or image abnormal breast tissue. In an example, components such as a power source, data processor, data transmitter/detector, and pump can be located on the posterior portion (e.g. the back strap) of a bra.

In an example, a device can be separate from a bra. In an example, this device can be a bra insert. In an example, this device can be removably inserted into the cup on a bra. In an example, this device can be removably inserted into a pocket, pouch, or other opening in a cup on a bra. In an example, this device can be removably attached to the concave interior of a cup of a bra. In an example, this device can be inserted between a bra cup and a breast. In an example, this device can be placed on a person's breast and then covered by a bra, wherein pressure from the bra cup causes the device to become (more) concave and conform to the shape of the breast.

In an example, a device can be a bra insert which is inserted between the cup of a conventional bra and a breast. In an example, a device can be an insert which is placed between the cup of a specialized bra and a breast. In an example, a system can comprise a specialized bra with one or more pockets in cups into which a concave insert with optical sensors is removably inserted. In an example, a system can comprise a specialized bra with attachment mechanisms (e.g. hook and loop fabric, snap, or clip) on the interior of cups to which an insert with optical sensors is removably attached. In an example, a bra insert can be inserted into a right-side cup of a smart bra in a first orientation and inserted into a left-side cup of a smart bra in a second (e.g. reflected) orientation.

In an example, this device can be embodied in a flexible patch, bandage, or sticker which is attached to a breast. In an example, this device can be embodied in a patch or bandage which is gently adhered to a breast. In an example, the perimeter of this device which is closest to the chest wall can be gently adhered to the chest wall. In an example, this device can be gently adhered to the chest wall—encompassing the Auxiliary Tail of Spence, the upper outer quadrant, the upper inner quadrant, the lower inner quadrant, and the lower outer quadrant of a breast. In an example, this device can further comprise an adhesive ring which is gently adhered to the chest wall where a breast is attached to the chest wall—encompassing the Auxiliary Tail of Spence, the upper outer quadrant, the upper inner quadrant, the lower inner quadrant, and the lower outer quadrant of the breast.

In an example, the interior layer of this device can be gently adhesive. Mild adhesion can help to keep the device in the same position relative to breast tissue during an optical, even during expansion of the expandable components and/or respiratory movement. In an example, a device can further comprise an array of small-scale suction elements on its interior. These small-scale suction elements can help to keep the cup in the same position relative to breast tissue during an optical scan, even during expansion of the expandable components and/or respiratory movement.

In an example, the perimeter of a device can also be coated with gentle adhesive to gently engage tissue close to the chest wall to keep light emitters as close as possible to the chest wall. This helps to image tissue which is as close to the chest wall as possible in addition to the main body of the breast. In an example, this device can further comprise disposable adhesive strips (or rings) which are removably attached to the inner layer of the device and to breast tissue near the chest wall in order to gently engage tissue close to the chest wall. This enables light emitters to be as close as possible to the chest wall. In an example, there can be a first disposable adhesive strip (e.g. half-ring) which is attached to the device on one side of an oblique virtual plane and a second disposable adhesive strip (e.g. half-ring) which is attached to the device on the opposite side of the oblique virtual plane.

In an example, this device can have a concave shape which fits over a breast. In an example, this device can have a substantially planar shape before being worn on a breast and is changed into convex shape by pressure from a bra cup as it worn on a breast. In an example, a cross-section of this device can have a teardrop cross-sectional shape. In an example, the perimeter of a device which is closest to the chest wall can have a teardrop shape. A teardrop shape is better than a radially-symmetric (e.g. circular) shape for encompassing the Auxiliary Tail of Spence and fully spanning where the upper outer quadrant connects to the chest wall. This is a design advantage over bra cups and/or bra inserts with a hemispherical shape (or other shape with a radially-symmetric perimeter closest to the chest wall. Bra cups and/or bra inserts with hemispherical shapes may be less effective at encompassing the Auxiliary Tail of Spence and full spanning where the upper outer quadrant connects to the chest wall.

In an example, the apex of a teardrop shape of this device can span the Auxiliary Tail of Spence. In an example, a teardrop-shaped cross-section of this device can have a shape whose two-dimensional parametric equation is X=cos(T) and Y=[sin(T)][sin M(T/2)]. In an alternative example, a cross-section of this device can have an elliptical or oval shape. In an example, a cross-section of this device can have a paisley shape. In an example, portions of this device which cover the upper inner, lower inner, and lower outer quadrants of the breast can have quarter-circle (e.g. quarter pie slice) cross-sectional perimeters. In an example, the portion of the device which covers the upper outer quadrant and the Auxiliary Tail of Spence can have a quadrilateral cross-sectional perimeter.

In an example, this device can be oriented around a virtual plane, with one set of optical and expandable components on one side of this virtual plan and a second set of optical and expandable components on the opposite side of this virtual. In an example, this virtual plan can be an oblique virtual plane. In an example, a virtual plane which separates two parts of an air-gap-reducing layer, an optical layer, and/or an expandable layer can be an oblique virtual plane which is neither horizontal (e.g. axial) nor vertical (e.g. sagittal). In an example, an oblique virtual plane can be created by 45-degree rotation of a horizontal (e.g. axial) or vertical (e.g. sagittal). In an example, an oblique virtual plane can intersect the upper outer quadrant (and/or the Auxiliary Tail of Spence) and the lower inner quadrant of a breast. In another example, a virtual plane can intersect the upper inner quadrant and the lower outer quadrant of a breast. In alternative examples, a virtual plane can be horizontal (e.g. axial) or vertical (e.g. sagittal).

In an example, an air-gap-reducing layer of this device can be transparent. In an example, an air-gap-reducing layer can enable light from light emitters in the optical layer to enter breast tissue with minimal refraction or scattering from air gaps. In an example, an air-gap-reducing layer can have optical characteristics like those of breast tissue. In an example, an air-gap-reducing layer can have one or more optical parameters (e.g. optical absorption coefficient, optical scattering coefficient, and/or anisotropy factor) a value which is within plus or minus 20% of the mean value for normal breast tissue.

In an example, an air-gap-reducing layer can one or more optical parameters (e.g. optical absorption coefficient, optical scattering coefficient, and/or anisotropy factor) which are each within one standard deviation of the mean parameter value for normal breast tissue. In an example, an air-gap-reducing layer can have an optical absorption coefficient with a value like the average value for normal breast tissue. In an example, an air-gap-reducing layer can have an optical scattering coefficient with a value like the average value for normal breast tissue. In an example, an air-gap-reducing layer can have an anisotropy factor with a value like that of breast tissue.

In an example, an air-gap-reducing layer can have an optical absorption coefficient with a value within plus or minus 20% of the average value for normal breast tissue. In an example, an air-gap-reducing layer can have an optical scattering coefficient with a value within plus or minus 20% of the average value for normal breast tissue. In an example, an air-gap-reducing layer can have an anisotropy factor with a value like that of breast tissue. In an example, an air-gap-reducing layer can have an optical absorption coefficient with a value within one standard deviation of the mean value (range) for normal breast tissue. In an example, an air-gap-reducing layer can have an optical scattering coefficient with a value within one standard deviation of the mean value (range) for normal breast tissue.

In an example, an air-gap-reducing layer can be sufficiently soft, compressible, elastomeric, and/or flexible to conform to the shape of the breast, thereby reducing air gaps between the device and the breast. In an example, an air-gap-reducing layer can be sufficiently soft, compressible, elastomeric, and/or flexible to conform to the shape of the breast under gentle pressure from the cup of a bra. In an example, an air-gap-reducing layer can have a Shore durometer level which is less than 30. In an example, an air-gap-reducing layer can have a Shore durometer level between 5 and 30. In an example, an air-gap-reducing layer can have a Young's modulus between 0.50 and 4.00.

In an example, an air-gap-reducing layer can contain a gel. In an example, an air-gap-reducing layer can be made from a xerogel. In an example, an air-gap-reducing layer can be made from a silicon composite. In an example, an air-gap-reducing layer can be made from an alginate. In an example, an air-gap-reducing layer can be made from a hydrogel. In an example, an air-gap-reducing layer can be made from a gelatin. In an example, an air-gap-reducing layer can be made from fibrin. In an example, an air-gap-reducing layer can be made from a starch. In an example, an air-gap-reducing layer can be made from chitosan. In an example, an air-gap-reducing layer can be made from a cryogel. In an example, an air-gap-reducing layer can be made from xanthan gum. In an example, an air-gap-reducing layer can be made from an aerogel. In an example, an air-gap-reducing layer can be made from collagen.

In an example, an air-gap-reducing layer can be made from Polyvinyl Alcohol (PVA). In an example, an air-gap-reducing layer can be made from Polyethylene Glycol (PEG). In an example, an air-gap-reducing layer can be made from Polymethyl Methacrylate (PMMA). In an example, an air-gap-reducing layer can be made from a copolymeric polymer gel. In an example, an air-gap-reducing layer can be made from Polyurethane (PU). In an example, an air-gap-reducing layer can be made from Polyvinyl Chloride (PVC). In an example, an air-gap-reducing layer can be made from Poly-acrylo-nitrile (PAN). In an example, an air-gap-reducing layer can be made from Poly-vinyl Chloride Plastisol (PVCP).

In an example, an air-gap-reducing layer can be made from Poly-vinyl Pyrrolidone (PVP). In an example, an air-gap-reducing layer can be made from polyamino acid. In an example, an air-gap-reducing layer can be made from Poly-di-methyl-siloxane (PDMS). In an example, an air-gap-reducing layer can be made from a homopolymeric polymer gel. In an example, an air-gap-reducing layer can be made from Poly-hydroxy-ethyl-methyl Acrylate (PHEMA). In an example, an air-gap-reducing layer can be made from an interpenetrating polymer gel. In an example, an air-gap-reducing layer can be made from Polyacrylic Acid (PA). In an example, an air-gap-reducing layer can be made from a PDMS-hydrogel composite.

In an example, an air-gap-reducing layer can be made with a transparent elastomeric material. In an example, an air-gap-reducing layer can be made with a silicone-based material such as polydimethylsiloxane (PDMS). In an example, an air-gap-reducing layer can comprise an acrylic elastomer. In an example, an air-gap-reducing layer can comprise polyethylene terephthalate (PET). In an example, an air-gap-reducing layer of this device can be the most elastic and most air-gap-reducing layer of a device. In an example, an air-gap-reducing layer can be between 0.5 mm and 2 mm thick. In another example, this layer can be between 2 mm and 8 mm thick.

In an example, a first part of an air-gap-reducing layer can be on one side of a virtual plane and a second part of the air-gap-reducing layer can be on the opposite side of the virtual plane. In an example, the perimeter of a first part of an air-gap-reducing layer on one side of a virtual plane can have a half-ring or horseshoe shape. In an example, the perimeter of a second part of the air-gap-reducing layer on an opposite side of the virtual plane can also have a half-ring or horseshoe shape, wherein the shape of the second part is symmetric to the shape of the first part (reflected across the virtual plane between the two parts). In an example, these two parts can be separate from each other (e.g. not continuous). In another example, these two parts can be part of a continuous layer.

In an example, a first part of an air-gap-reducing layer on a first side of a virtual plane can be pushed closer to a second part of an air-gap-reducing layer on a second (e.g. opposite) side of the virtual plane when the expandable layer is expanded and the device is changed from its first (unexpanded) configuration to its second (expanded) configuration. In an example, a first part of an air-gap-reducing layer on a first side of a virtual plane can be pushed closer to a second part of an air-gap-reducing layer on a second (e.g. opposite) side of the virtual plane when the expandable layer is expanded and the device is changed from its first (unexpanded) configuration to its second (expanded) configuration, thereby compressing the breast between the first and second parts.

In an example, a first part of an air-gap-reducing layer can span the lower inner quadrant, the lower outer quadrant, the upper outer quadrant, and the Auxiliary Tail of Spence of a breast. In an example, a second part of the air-gap-reducing layer can also span the span the lower inner quadrant, the lower outer quadrant, the upper outer quadrant, and the Auxiliary Tail of Spence, although the two parts are not continuous.

In an example, a part of the air-gap-reducing layer can have a first degree of concavity when the device is in the first (unexpanded) configuration and a second degree of concavity with the device is in the second (expanded) configuration, wherein the second degree is less than the first degree. In an example, two parts of the air-gap-reducing layer on opposite sides of the virtual plane can be closer to parallel when the device is in the second configuration than when the device is in the first configuration. In an example, central portions of first and second parts of an air-gap-reducing layer can be moved a greater distance than non-central portions of the first and second parts when an expanding layer is expanded and the device is changed from its first configuration to its second configuration.

In an example, the thickness, elasticity, and/or compressibility of an air-gap-reducing layer can be adjusted in order to better conform the layer to the shape and size of a breast. In an example, the thickness, elasticity, and/or compressibility of an air-gap-reducing layer can be adjusted by pumping fluid (or gel) into the layer or out of the layer. In an example, the thickness, elasticity, and/or compressibility of an air-gap-reducing layer can be adjusted by changing the pressure level of fluid (or gel) in the layer. In an example, the thickness, elasticity, and/or compressibility of an air-gap-reducing layer can be adjusted by inflation. In an example, the thickness, elasticity, and/or compressibility of an air-gap-reducing layer can be adjusted by changing the pressure of a gas inside the layer.

In an example, a device can have an optical layer. This optical layer can comprise a plurality of light emitters and light detectors. In an example, light from the light emitters can be received by the light detectors after it has passed through breast tissue. In an example, changes in light caused by transmission through breast tissue can be analyzed to image breast tissue and/or detect breast cancer. In an example, changes in amplitude and/or spectrum of light from the light emitters which are caused by transmission of the light through breast tissue can be analyzed to image breast tissue and/or detect breast cancer. In an example, changes in amplitude and/or spectrum of light from the light emitters which are caused by reflection of the light by breast tissue and/or transmission of the light through breast tissue can be analyzed to image breast tissue and/or detect breast cancer.

In an example, a light emitter in an optical layer can be a Light Emitting Diode (LED). In an example, a light emitter can be a Laser LED. In an example, a light emitter can be a Super-Luminescent Light Emitting Diode (SLED). In an example, a light emitter can be a Single Photon Avalanche Diode (SPAD). In an example, a light emitter can be an Organic Light Emitting Diode (OLED). In an example, a light emitter can be a Resonant Cavity Light Emitting Diode (RCLED). In an example, a light emitter can be a Vertical Cavity Surface Emitting Laser (VCSEL).

In an example, a light emitter can be a Quantum Dot LED (QLED). In an example, a light emitter can be a Phosphorescent OLED (PHOLED). In an example, a light emitter can be a Light-Emitting Electrochemical Cell (LEC). In an example, a light emitter can be a MicroLED. In an example, a light emitter can be a Nanoscale LED. In an example, a light emitter can be an Active Matrix Organic Light-Emitting Diode (AMOLED). In an example, a light emitter can be a Monochromatic LED (MLED). In an example, a light emitter can be a Multi-Wavelength Light Emitting Diode (MWLED). In an example, a light emitter can be an Organic Photovoltaic (OPV). In an example, a light emitter can be a Side-Emitting Polymer Optical Fiber (SEPOF).

In an example, light emitters in an optical layer can emit coherent light. In an example, light emitters in an optical layer can emit light in pulses. In an example, light emitters in an optical layer can emit near-infrared light. In an example, light emitters in an optical layer can emit polarized light.

In an example, light emitters in an optical layer can be part of yarns or fibers which are woven to make a fabric or textile used to make this device. In an example, light emitters can be attached to a fabric or textile which is used to make this device. In an example, light emitters can be printed on a fabric or textile. In an example, light emitters can be made by 3D printing. In an example, light emitters can be encapsulated with a waterproof coating for protection from moisture. In an example, light emitters can be encapsulated in acrylic material for protection from moisture. In an example, optical components can be separated from the surface of a breast by an air-gap-reducing layer which transmits light, but protects the optical components when the device is washed.

In an example, one or more light emitters in an optical layer can be Light Emitting Diodes (LEDs). In an example, one or more light emitters in an optical layer can be near-infrared light emitters. In an example, one or more light emitters in an optical layer can be pulsatile lasers. In an example, one or more light emitters in an optical layer can be a green-light laser. In an example, one or more light emitters in an optical layer can be red-light lasers. In an example, one or more light emitters in an optical layer can be Resonant Cavity Light Emitting Diodes (RCLEDs). In an example, one or more light emitters in an optical layer can be Single Photon Avalanche Diodes (SPADs).

In an example, one or more light emitters in an optical layer can be MicroLEDs. In an example, one or more light emitters in an optical layer can be Super-Luminescent Light Emitting Diodes (SLEDs). In an example, one or more light emitters in an optical layer can be tunable LEDs. In an example, one or more light emitters in an optical layer can be ultraviolet light emitters. In an example, one or more light emitters in an optical layer can be monochromatic LEDs.

In an example, one or more light emitters in an optical layer can be multi-wavelength lasers. In an example, one or more light emitters in an optical layer can be Active Matrix Organic Light-Emitting Diodes (AMOLEDs). In an example, one or more light emitters in an optical layer can be infrared light emitters. In an example, one or more light emitters in an optical layer can be an Organic Light Emitting Diodes (OLEDs). In an example, one or more light emitters in an optical layer can be lasers. In an example, one or more light emitters in an optical layer can be a Light-Emitting Electrochemical Cells (LECs). In an example, one or more light emitters in an optical layer can be coherent light emitters.

In an example, light emitters in an optical layer can emit light with one or more wavelengths, within the range of 600 to 1100 nm. In an example, light emitters in an optical layer can emit light with one or more wavelengths, within the range of 600 to 1100 nm. In an example, a light emitter in an optical layer can emit light at different wavelengths over time, within the range of 600 to 1100 nm. In an example, a light emitter in an optical layer can emit light at different wavelengths over time, within the range of 600 to 1100 nm.

In an example, a light emitter in an optical layer can emit light at different wavelengths over time selected from the group consisting of: 600, 650, 660, 680, 690, 750, 775, 780, 785, 800, 808, 810, 830, 850, and 1000 nm. In an example, different light emitters in an optical layer can emit light with different wavelengths, within the range of 600 to 1100 nm. In an example, different light emitters in an optical layer can emit light with different wavelengths, within the range of 600 to 1100 nm. In an example, different light emitters in an optical layer can emit light with different wavelengths, within the range of 600 to 1100 nm.

In an example, different light emitters in an optical layer can emit light with different wavelengths selected from the group consisting of: 600, 650, 660, 680, 690, 750, 775, 780, 785, 800, 808, 810, 830, 850, and 1000 nm. In an example, light emitters in an optical layer can emit light at different wavelengths selected from the group consisting of: 600, 650, 660, 680, 690, 750, 775, 780, 785, 800, 808, 810, 830, 850, and 1000 nm. In an example, light emitters in an optical layer can emit light at different wavelengths over time, within the range of 600 to 1100 nm.

In an example, light emitters in an optical layer can emit light at different wavelengths over time selected from the group consisting of: 600, 650, 660, 680, 690, 750, 775, 780, 785, 800, 808, 810, 830, 850, and 1000 nm. In an example, light emitters in an optical layer can emit light at different wavelengths over time, within the range of 600 to 1100 nm. In an example, a light emitter in an optical layer can emit light at different wavelengths over time, within the range of 600 to 1100 nm. In an example, light emitters in an optical layer can emit light at different wavelengths over time, within the range of 600 to 1100 nm. In an example, light emitters in an optical layer can emit light with one or more wavelengths, within the range of 600 to 1100 nm.

In an example, a first set of light emitters can emit light at a first frequency and/or wavelength (or in a first spectral range), a second set of light emitters can emit light at a second frequency and/or wavelength (or in a second spectral range), and a third set of light emitters can emit light at a third frequency and/or wavelength (or in a third spectral range). In an example, a first light emitter can emit light with a wavelength in the range of 650 to 750 nm at a first time; a second light emitter can emit light with a wavelength in the range of 750 nm to 850 nm at a second time; and a third light emitter can emit light with a wavelength in the range of 850 nm to 950 nm at a third time. In an example, a first light emitter can emit light with a wavelength in the range of 650 to 700 nm; a second light emitter can emit light with a wavelength in the range of 700 nm to 750 nm; and a third light emitter can emit light with a wavelength in the range of 750 nm to 800 nm.

In an example, a first set of light emitters can emit light at a first frequency and/or wavelength (or in a first spectral range) and a second set of light emitters can emit light at a second frequency and/or wavelength (or in a second spectral range). In an example, a first light emitter can emit light with a wavelength in the range of 600 to 900 nm; a second light emitter can emit light with a wavelength in the range of 900 nm to 1200 nm; and a third light emitter can emit light with a wavelength in the range of 1200 nm to 1500 nm. In an example, a first light emitter can emit light with a wavelength in the range of 600 to 700 nm at a first time; a second light emitter can emit light with a wavelength in the range of 700 nm to 800 nm at a second time; and a third light emitter can emit light with a wavelength in the range of 800 nm to 900 nm at a third time. In an example, a first light emitter can emit light with a wavelength in the range of 650 to 750 nm; a second light emitter can emit light with a wavelength in the range of 750 nm to 850 nm; and a third light emitter can emit light with a wavelength in the range of 850 nm to 950 nm.

In an example, a first light emitter can emit light with a wavelength in the range of 600 to 700 nm; a second light emitter can emit light with a wavelength in the range of 700 nm to 800 nm; and a third light emitter can emit light with a wavelength in the range of 800 nm to 900 nm. In an example, a first light emitter can emit light with a wavelength in the range of 650 to 700 nm; a second light emitter can emit light with a wavelength in the range of 700 nm to 750 nm; and a third light emitter can emit light with a wavelength in the range of 750 nm to 800 nm. In an example, a first light emitter can emit intensity or amplitude modulated light into the breast with a wavelength in the range of 650 to 750 nm; a second light emitter can emit intensity or amplitude modulated light with a wavelength in the range of 750 nm to 850 nm; and a third light emitter can emit intensity or amplitude modulated light with a wavelength in the range of 850 nm to 950 nm.

In an example, a first light emitter can emit light with a wavelength in the range of 600 to 800 nm at a first time; a second light emitter can emit light with a wavelength in the range of 800 nm to 1000 nm at a second time; and a third light emitter can emit light with a wavelength in the range of 1000 nm to 1200 nm at a third time. In an example, a first light emitter can emit light with a wavelength in the range of 600 to 800 nm; a second light emitter can emit light with a wavelength in the range of 800 nm to 1000 nm; and a third light emitter can emit light with a wavelength in the range of 1000 nm to 1200 nm. In an example, a first light emitter can emit light with a wavelength in the range of 600 to 900 nm at a first time; a second light emitter can emit light with a wavelength in the range of 900 nm to 1200 nm at a second time; and a third light emitter can emit light with a wavelength in the range of 1200 nm to 1500 nm at a third time.

In an example, a first light emitter can emit light with a wavelength in the range of 600 to 900 nm and a second light emitter can emit light with a wavelength in the range of 900 nm to 1200 nm. In an example, a first light emitter can emit light with a wavelength in the range of 650 to 750 nm; a second light emitter can emit light with a wavelength in the range of 750 nm to 850 nm; and a third light emitter can emit light with a wavelength in the range of 850 nm to 950 nm. In an example, a first light emitter can emit light with a wavelength in the range of 600 to 900 nm at a first time and a second light emitter can emit light with a wavelength in the range of 900 nm to 1200 nm at a second time. In an example, a light emitter can emit coherent light.

In an example, a light emitter can emit light pulses. In an example, light emitters can emit short pulses of light. In another example, light emitters can be continuous wave light emitters. In an example, a light emitter can emit light with a variable frequency. In an example, a first set of light emitters can emit light at a first intensity or amplitude level (or at a first time) and a second set of light emitters can emit light at a second intensity or amplitude level (or at a second time). In an example, a light emitter can emit light with a wavelength and/or frequency which changes over time. In an example, a light emitter can emit light with a wavelength and/or frequency which changes in a repeated cyclical pattern over time.

In an example, a light emitter can emit light at a frequency and/or wavelength which varies over time. In an example, a light emitter can emit light within a spectral range which varies over time. In an example, a light emitter can emit light at different wavelengths at different times. In an example, different light emitters in an array of light emitters can emit light at different times. In an example, light can be emitted from light emitters in very short pulses. In an example, light emitters on a cup can emit frequency and/or wavelength modulated light.

In an example, light emitters in a first quadrant can emit (a pulse of) light at a first time and light emitters in a second quadrant can emit (a pulse of) light at a second time. In an example, a light emitter can emit light at an angle and/or along a focal vector which varies over time. In an example, a light emitter can emit light at different wavelengths at different times. In an example, a first light emitter at a first location can emit (a pulse of) light at a first time and a second light emitter at a second location can emit (a pulse of) light at a second time. In an example, light emitters on a right side of a device can emit (a pulse of) light at a first time and light emitters on the left side of a device can emit (a pulse of) light at a second time, or vice versa. In an example, a light emitter can be a laser with a narrow pulse width.

In an example, a light emitter can emit light via Alternating Current Electroluminescence (ACEL). In an example, a light emitter can emit a first pulse of light with a first duration followed by a second pulse of light with a second duration, wherein the second duration is greater than the first duration. In an example, a light emitter can emit light at a constant frequency and/or in a constant spectral range. In an example, light emitters can emit light at a frequency and/or wavelength which varies over time. In an example, a first light emitter can emit a pulse of light with a first duration and a second light emitter can emit a pulse of light with a second duration, wherein the second duration is greater than the first duration.

In an example, light emitters on the top half of a cup can emit (a pulse of) light at a first time and light emitters on the bottom half of the cup can emit (a pulse of) light at a second time, or vice versa In an example, light emitters one a first of light emitters can emit (a pulse of) light at a first time and light emitters on a second ring of light emitters can emit (a pulse of) light at a second time. In an example, light emitters can all emit a pulse of light at the same time. In an example, a light emitter can emit intensity or amplitude-modulated light. In an example, light emission from light emitters can be multiplexed.

In an example, light emitters in an optical layer can be arranged in (e.g. distributed along) rings. In an example, light emitters in an optical layer can be arranged in (e.g. distributed along) nested (e.g. concentric) rings. In an example, light emitters in an optical layer can be arranged in a hub-and-spoke configuration. In an example, light emitters in an optical layer can be arranged in radial spokes. In an example, light emitters in an optical layer can be arranged in (e.g. distributed along) an orthogonal mesh, grid, and/or matrix. In an example, light emitters in an optical layer can be arranged in (e.g. distributed along) a hexagonal (e.g. honeycomb) mesh, grid, and/or matrix. In an example, an optical layer can further comprise a plurality of mirrors which change the vectors of light rays from a plurality of light emitters.

In an example light emitters in an optical layer can be configured in a honeycomb array (e.g. hexagonal grid or mesh). In an example light emitters in an optical layer can be configured in concentric (e.g. nested) rings. In an example light emitters in an optical layer can be configured in a half-helical array. In an example light emitters in an optical layer can be configured in a hub-and-spoke array. In an example light emitters in an optical layer can be configured along undulating (e.g. sinusoidal) rings around a breast. In an example light emitters in an optical layer can be configured in a star-burst array. In an example light emitters in an optical layer can be configured in concentric (e.g. nested) half rings.

In an example light emitters in an optical layer can be configured in a helical array. In an example light emitters in an optical layer can be configured in a spiral array. In an example light emitters in an optical layer can be configured in an orthogonal matrix (e.g. quadrilateral grid or mesh). In an example light emitters in an optical layer can be configured in evenly spaced along latitudinal lines around a breast. In an example light emitters in an optical layer can be configured along undulating (e.g. sinusoidal) pathways. In an example light emitters in an optical layer can be configured in a checkerboard array. In an example light emitters in an optical layer can be configured in evenly spaced along longitudinal lines around a breast.

In an example, the density of light emitters in a device can be greater (and/or the distance between light emitters can be less) for portions of the optical layer which are farther from the center (and/or apex) of the device than for portions of the optical layer which are closer to the center (and/or apex) of the device. In an example, the density of light emitters in a device can be less (and/or the distance between light emitters can be greater) for portions of the optical layer which are farther from the center (and/or apex) of the device than for portions of the optical layer which are closer to the center (and/or apex) of the device.

In an example, the density of light emitters in the portion of a device covering the upper outer quadrant of a breast can be greater (and/or the distance between light emitters can be less) than for the portion of the device covering the lower inner quadrant of the breast. In an example, the density of light emitters in the portion of a device covering the upper outer quadrant of a breast can be less (and/or the distance between light emitters can be greater) than for the portion of the device covering the lower inner quadrant of the breast. In an example, the density of light emitters in the portion of a device covering the upper outer quadrant and Auxiliary Tail of Spence of a breast can be greater (and/or the distance between light emitters can be less) than for the portion of the device covering the lower inner quadrant of the breast.

In an example, light emitters in the portion of the device which covers the upper outer quadrant of the breast can be closer together than light emitters in other portions of the device. In an example, light emitters in the portion of the device which covers the lower outer quadrant of the breast can be closer together than light emitters in other portions of the device. In an example, light emitters in the portion of the device which covers the upper outer quadrant and the Auxiliary Tail of Spence of the breast can be closer together than light emitters in other portions of the device. In an example, light emitters can be farther apart toward the apex of a cup and closer together toward the periphery of the cup.

In an example, light emitters which are closer to the apex of a cup can be farther apart than light emitters which are farther from the apex of a cup. In an example, light emitters which are closer to an oblique virtual plane spanning the upper outer quadrant and the lower inner quadrant can be farther apart than light emitters which are farther from this virtual plane. In an example, light emitters which are farther from the apex of a cup can be farther apart than light emitters which are closer to the apex of a cup. In an example, light emitters which are farther from the chest wall can be farther apart than light emitters which are closer to the chest wall. In an example, light emitters which are closer to the chest wall can be farther apart than light emitters which are farther from the chest wall. In an example, light emitters which are farther from an oblique virtual plane spanning the upper outer quadrant and the lower inner quadrant can be farther apart than light emitters which are closer to this virtual plane.

In an example, a light emitter can be oriented to emit light along a vector which is substantially perpendicular to the closest surface of a breast. In an example, a light emitter can be positioned so as to emit light toward a particular light detector. In an example, a light emitter can be positioned so as to emit light along a vector which is substantially perpendicular to a breast surface and/or directed toward a particular light detector. In an example, a light emitter can emit a radially-rotating beam of light. In an example, angles between the focal vectors of light beams emitted from light emitters and the surface of a breast or cup can vary with the distance of the light emitters from the apex of a device. In an example, a light emitter can be positioned so as to emit light along a vector which is substantially perpendicular to a breast or device surface.

In an example, an optical component can include an electromagnetic actuator which changes the angle and/or focal vector of light emission over time. In an example, angles between the focal vectors of light beams emitted from light emitters and the surface of a breast or device can vary with the distance of those light emitters from the apex of a device. In an example, angles between the focal vectors of light emitted from light emitters and the surface of a breast or device can increase with the distance of the light emitters from the apex of the concave surface of a breast or device. In an example, different light emitters can emit light at different wavelengths. In an example, different light emitters in a ring can emit light at different wavelengths. In an example, different light emitters in an array can emit light at different wavelengths.

In an example, an optical layer can distribute light from light emitters (e.g. LEDs) across a concave inner surface via a plurality of optical fibers (e.g. light-conducting fibers, tubes, channels, or threads). In an example, an optical layer can comprise a plurality of locations on a plurality of optical fibers (e.g. fibers, tubes, channels, or threads) which emit light from an LED located elsewhere. In an example, optical fibers can transmit light from an optical layer which originates from light sources (e.g. LEDs) outside the optical layer. In an example, an optical layer can comprise a plurality of endpoints (or side locations) on a plurality of optical fibers which emit light, wherein these optical fibers transmit this light into the optical layer from light sources (e.g. LEDs) outside the optical layer. In an example, an optical layer can comprise a plurality of endpoints (or side locations) on a plurality of optical fibers which emit light, wherein these optical fibers transmit this light from light sources (e.g. LEDs) around the chest-wall perimeter of the optical layer.

In an example, an optical layer can further comprise optical fibers which guide light from a plurality of light emitters to a plurality of light-exiting points. In an example, an optical layer can further comprise optical fibers which guide light to light-exiting points which are distributed around the interior concavity of the device. In an example, an optical layer can further comprise optical fibers which emit light from their ends at a plurality of light-exiting points around the interior concavity of the device. In an example, an optical layer can further comprise elastic optical fibers which guide light to light-exiting points. In an example, an optical layer can further comprise undulating (e.g. sinusoidal) optical fibers which guide light to light-exiting points.

In an example, an optical layer of this device can be made from one or more materials selected from the group consisting of: Poly-Di-Methyl-Siloxane (PDMS), Poly-Ethylene-Di-Oxy-Thiophene Poly-Styrene Sulfonate (PEDOT:PSS), nanotubes, two-dimensional nanomaterial, Molybdenum Disulfide (MD), Thermoplastic Poly-Urethane (TPU), and an multi-conjugated organic semiconductor.

In an example, an optical layer of this device can be made from one or more materials selected from the group consisting of: Poly-Imide (PI), parylene, a perovskite, Poly-Lactic Acid (PLA), acrylic, polymer and non-fullerene acceptor composite/hybrid, black phosphorus, Poly-Styrene (PS), Poly-Ethylene Glycol Di-Acrylate (PEGDA), cellulose, Poly-Urethane (PU), chitosan, quantum dots, colloidal quantum, Shape Memory Photonic Crystal Fiber (SMPF), and organic material.

In an example, an optical layer of this device can be made from one or more materials selected from the group consisting of: Zinc Oxide Nanoparticles, one-dimensional nanomaterial, zero-dimensional nanomaterial, Poly-Ethylene Naphthalate (PEN), Poly-Ethylene Terephthalate (PET), a carbon nanomaterial, Poly Lactic-co-Glycolic Acid (PLGA), a hydrogel, Poly-Glycolic Acid (PGA), Poly Methyl Meth-Acrylate (PMMA), Poly-Ethlylene Terephthalate (PET), nanocrystals, Transition Metal Dichalcogenide (TMD), organic-inorganic composite/hybrid material, inorganic graphene, silk, graphene, and silicone rubber.

In an example, a light detector can be a photodetector. A photodetector absorbs photons and generates electrical current—converting light to electricity. In an example, a light detector in an optical layer can be selected from the group consisting of: photodiode, photomultiplier, photoconductor, avalanche photodiode, organic photodiode, organic photo-detector, silicon photodiode, photon multiplier, polarization-sensitive photodetector, Charge-Coupled Device (CCD), and Silicon Photo-Multiplier (SiPM).

In an example, a light detector can be a photoreceptor. In an example, a light detector can be a photodiode. In an example, a light detector can be a photoconductor. In an example, a light detector can be a thin-film photoreceptor. In an example, a light detector can be an organic phototransistor. In an example, a light detector can comprise a fast-gated detector. In an example, a light detector can have an organic photoactive channel layer, a dielectric layer, and electrodes. In an example, a light detector can be an organic photodiode. In an example, a light detector can be an avalanche photo diode (APDs) or PIN photodiode.

In an example, a light detector can be made with polydimethylsiloxane (PDMS) or another silicone-based polymer. In an example, a light detector can be made with an acrylic elastomer. In an example, a light detector can be selected from the group consisting of: photodetector, photoresistor, avalanche photodiode (APD), charge-coupled device (CCD), complementary metal-oxide semiconductor (CMOS), infrared detector, infrared photoconductor, infrared photodiode, light dependent resistor (LDR), optoelectric sensor, photoconductor, photodiode, photomultiplier, and phototransistor.

In an example, light detectors in an optical layer can be arranged in (e.g. distributed along) rings. In an example, light detectors in an optical layer can be arranged in (e.g. distributed along) nested (e.g. concentric) rings. In an example, light detectors in an optical layer can be arranged in a hub-and-spoke configuration. In an example, light detectors in an optical layer can be arranged in radial spokes. In an example, light detectors in an optical layer can be arranged in (e.g. distributed along) an orthogonal mesh, grid, and/or matrix. In an example, light detectors in an optical layer can be arranged in (e.g. distributed along) a hexagonal (e.g. honeycomb) mesh, grid, and/or matrix.

In an example light detectors in an optical layer can be configured in a honeycomb array (e.g. hexagonal grid or mesh). In an example light detectors in an optical layer can be configured in concentric (e.g. nested) rings. In an example light detectors in an optical layer can be configured in a half-helical array. In an example light detectors in an optical layer can be configured in a hub-and-spoke array. In an example light detectors in an optical layer can be configured along undulating (e.g. sinusoidal) rings around a breast. In an example light detectors in an optical layer can be configured in a star-burst array. In an example light detectors in an optical layer can be configured in concentric (e.g. nested) half rings.

In an example light detectors in an optical layer can be configured in a helical array. In an example light detectors in an optical layer can be configured in a spiral array. In an example light detectors in an optical layer can be configured in an orthogonal matrix (e.g. quadrilateral grid or mesh). In an example light detectors in an optical layer can be configured in evenly spaced along latitudinal lines around a breast. In an example light detectors in an optical layer can be configured along undulating (e.g. sinusoidal) pathways. In an example light detectors in an optical layer can be configured in a checkerboard array. In an example light detectors in an optical layer can be configured in evenly spaced along longitudinal lines around a breast.

In an example, the density of light detectors in a device can be greater (and/or the distance between light detectors can be less) for portions of the optical layer which are farther from the center (and/or apex) of the device than for portions of the optical layer which are closer to the center (and/or apex) of the device. In an example, the density of light detectors in a device can be less (and/or the distance between light detectors can be greater) for portions of the optical layer which are farther from the center (and/or apex) of the device than for portions of the optical layer which are closer to the center (and/or apex) of the device.

In an example, the density of light detectors in the portion of a device covering the upper outer quadrant of a breast can be greater (and/or the distance between light detectors can be less) than for the portion of the device covering the lower inner quadrant of the breast. In an example, the density of light detectors in the portion of a device covering the upper outer quadrant of a breast can be less (and/or the distance between light detectors can be greater) than for the portion of the device covering the lower inner quadrant of the breast. In an example, the density of light detectors in the portion of a device covering the upper outer quadrant and Auxiliary Tail of Spence of a breast can be greater (and/or the distance between light detectors can be less) than for the portion of the device covering the lower inner quadrant of the breast.

In an example, light detectors in the portion of the device which covers the upper outer quadrant of the breast can be closer together than light detectors in other portions of the device. In an example, light detectors in the portion of the device which covers the lower outer quadrant of the breast can be closer together than light detectors in other portions of the device.

In an example, light detectors in the portion of the device which covers the upper outer quadrant and the Auxiliary Tail of Spence of the breast can be closer together than light detectors in other portions of the device. In an example, light detectors can be farther apart toward the apex of a cup and closer together toward the periphery of the cup. In an example, in an example, there can be equal numbers of light emitters on either side of an oblique virtual plane which spans from the Auxiliary Tail of Spence to the lower inner quadrant of a breast. In an example, light emitters can all be on the same side of this oblique virtual plane.

In an example, light emitters can be closer together in the upper-left quadrant of the cup on right breast, as compared to other quadrants on that cup. In an example, light emitters can be closer together in the upper-right quadrant of the cup on left breast, as compared to other quadrants on that cup. In an example, light emitters can be closer together toward the apex of a cup and farther apart toward the periphery of the cup. In an example, light emitters can be equally distributed on a given side (e.g. right or left, lower or upper) of a cup. In an example, light emitters can be farther apart toward the apex of a cup and closer together toward the periphery of the cup.

In an example, light detectors which are closer to the apex of a cup can be farther apart than light detectors which are farther from the apex of a cup. In an example, light detectors which are closer to an oblique virtual plane spanning the upper outer quadrant and the lower inner quadrant can be farther apart than light detectors which are farther from this virtual plane. In an example, light detectors which are farther from the apex of a cup can be farther apart than light detectors which are closer to the apex of a cup. In an example, light detectors which are farther from the chest wall can be farther apart than light detectors which are closer to the chest wall. In an example, light detectors which are closer to the chest wall can be farther apart than light detectors which are farther from the chest wall. In an example, light detectors which are farther from an oblique virtual plane spanning the upper outer quadrant and the lower inner quadrant can be farther apart than light detectors which are closer to this virtual plane.

In an example, light detectors in an optical layer can be made from one or more materials selected from the group consisting of: Poly-Ethylene Terephthalate (PET), a perovskite, Poly-Imide (PI), polymer and non-fullerene acceptor composite/hybrid, Transition Metal Dichalcogenide (TMD), one-dimensional nanomaterial, organic material, organic-inorganic composite/hybrid material, and Zinc Oxide Nanoparticles., Poly-Ethylene Naphthalate (PEN), zero-dimensional nanomaterial, colloidal quantum, Molybdenum Disulfide (MD), two-dimensional nanomaterial, a multi-conjugated organic semiconductor, and inorganic graphene.

In an example, a light detector can be made from silicon. In an example, a light detector can be made from semiconducting polymer. In an example, a light detector can be made from PEDOT:PSS. In an example, a light detector can be made from Germanium. In an example, a light detector can be made from carbon nanotubes. In an example, a light detector can be made from silver nanowires. In an example, a light detector can be flexible. In an example, a light detector can be made from polydimethylsiloxane (PDMS). In an example, a light detector can be made with polyethylene naphthalate. In an example, a light detector can be a flexible organic photodetector (OPD). In an example, a light detector can comprise a bicontinuous interpenetrating network of donor and acceptor materials.

In an example, this device can detect and/or image abnormal tissue by analyzing light transmission. In an example, a first part of an optical layer on a first side of a virtual plane can comprise light emitters and a second part of the optical layer on a second (e.g. opposite) side of the virtual plane can comprise light detectors. In an example, light emitted from the light emitters on the first side of the virtual plane can be received by light detectors on the second side of the virtual plane. In an example, changes in light transmitted from the first part of the optical layer to the second part of the optical layer through breast tissue can be analyzed to detect and/or image abnormal tissue. In an example, changes in the intensity and/or spectrum of light caused by its transmission through breast tissue from light emitters in the first part of the optical layer to light detectors in the second part of the optical layer through breast tissue can be analyzed to detect and/or image abnormal tissue.

In an example, this device can detect and/or image abnormal tissue by analyzing light reflection. In an example, a first part of an optical layer on a first side of a virtual plane can comprise both light emitters and light detectors. In an example, a first part of an optical layer on a second (e.g. opposite) side of a virtual plane can comprise both light emitters and light detectors. In an example, light emitted from the light emitters on a first side of the virtual plane can be received by light detectors on the first side of the virtual plane. In an example, changes in light reflected from breast tissue can be analyzed to detect and/or image abnormal tissue. In an example, both light transmitted through breast tissue and light reflected from breast tissue can be analyzed to detect and/or image abnormal tissue.

In an example, an optical layer can comprise light emitters and light detectors which are attached to, or integrated into, a flexible polymer substrate. In an example, an optical layer can comprise light emitters and light detectors which are attached to, or integrated into, an elastomeric substrate. In an example, an optical layer can comprise light emitters and light detectors which are attached to, or integrated into, an elastomeric polymer layer. In an example, an optical layer can comprise light emitters, light detectors, and electroconductive pathways which are attached to, or integrated into, an elastomeric polymer layer. In an example, an optical layer can comprise light emitters, light detectors, and undulating microwires which are attached to, or integrated into, an elastomeric polymer layer. In an example, an optical layer can comprise light emitters, light detectors, and electroconductive pathways which are printed on elastomeric substrate.

In an example, there can be a first average distance between light emitters and light detectors when a device is in its first (unexpanded) configuration and a second average distance between light emitters and light detectors when the device is in its second (expanded) configuration, wherein the second average distance is less than the first average distance. In an example, there can be a first maximum distance between light emitters and light detectors when a device is in its first (unexpanded) configuration and a second maximum distance between light emitters and light detectors when the device is in its second (expanded) configuration, wherein the second average distance is less than the first average distance.

In an example, an optical layer can comprise a plurality of optical modules, wherein each module includes at least one light emitter and at least one light detector. In an example, an optical module can include one light emitter and a plurality of light detectors. In an example, an optical module can include one light emitter and a plurality of light detectors distributed around the light emitter. In an example, an optical module can include one light emitter and a plurality of light detectors which are evenly-distributed around the light emitter.

In an example, an optical module can include one light emitter and at least four light detectors which are evenly-distributed around the light emitter. In an example, an optical module can include one light detector and a plurality of light emitters. In an example, an optical module can include one light detector and a plurality of light emitters distributed around the light detector. In an example, an optical module can include one light detector and a plurality of light emitters which are evenly-distributed around the light detector. In an example, an optical module can include one light detector and at least four light emitters which are evenly-distributed around the light detector.

In an example, an optical layer can comprise a plurality of light emitters around the chest-wall perimeter of the optical layer and a plurality of light detectors around the concave interior of the optical layer. In an example, an optical layer can comprise a plurality of light detectors around the chest-wall perimeter of the optical layer and a plurality of light emitters around the concave interior of the optical layer.

In an example, the distances between light emitters and detectors in a device can be greater for portions of the optical layer which are farther from the center (and/or apex) of the device than for portions of the optical layer which are closer to the center (and/or apex) of the device. In an example, the distances between light emitters and detectors in a device can be less for portions of the optical layer which are farther from the center (and/or apex) of the device than for portions of the optical layer which are closer to the center (and/or apex) of the device.

In an example, the distances between light emitters and detectors in the portion of a device covering the upper outer quadrant of a breast can be greater than for the portion of the device covering the lower inner quadrant of the breast. In an example, the distances between light emitters and detectors in the portion of a device covering the upper outer quadrant of a breast can be less than for the portion of the device covering the lower inner quadrant of the breast. In an example, the distances between light emitters and detectors in the portion of a device covering the upper outer quadrant and Auxiliary Tail of Spence of a breast can be greater than for the portion of the device covering the lower inner quadrant of the breast.

In an example, an optical layer can span all of the interior concavity of a device. In an example, an optical layer can span between 50% and 75% of the interior concavity of the device. In an example, an optical layer can span between 60% and 85% of the interior concavity of the device. In an example, an optical layer can span the entire perimeter of the device. In an example, an optical layer can span between 50% and 75% of the perimeter of the device. In an example, an optical layer can span between 60% and 85% of the perimeter of the device.

In an example, light emitters in an optical layer can receive electrical power through undulating (e.g. undulating, wavy, zigzag, and/or sinusoidal) wires (e.g. microwires or nanowires). In an example, light emitters in an optical layer can receive electrical power through undulating wires which are embedded in an elastomeric polymer. In an example, light emitters in an optical layer can receive electrical power through electroconductive channels in an elastomeric polymer. In an example, light emitters in an optical layer can receive electrical power through carbon nanotubes in an elastomeric polymer (e.g. PDMS). In an example, light emitters in an optical layer can receive electrical power through channels comprising an elastomeric polymer which has been embedded, impregnated, and/or coated with electroconductive material.

In an example, an optical layer can further comprise a plurality of elastic electroconductive pathways which are configured in a honeycomb (e.g. hexagonal) grid or mesh. In an example, an optical layer can further comprise a plurality of elastic electroconductive pathways which are configured in an undulating (e.g. sinusoidal) pattern. In an example, an optical layer can further comprise a plurality of elastic electroconductive pathways which are configured in nested (e.g. concentric) rings. In an example, an optical layer can further comprise a plurality of elastic electroconductive pathways which are configured in a radial strips which extend outward from the apex of a cup.

In an example, an optical layer can further comprise flexible electroconductive pathways which are in electrical communication with light emitters and light detectors. In an example, an optical layer can further comprise undulating (e.g. sinusoidal or zigzag) electroconductive pathways which are in electrical communication with light emitters and light detectors. In an example, light emitters can receive power from undulating (e.g. sinusoidal) electroconductive pathways in an optical layer. In an example, light emitters can receive power from undulating (e.g. sinusoidal) wires in an optical layer.

In an example, a device can further comprise optical shielding and/or barriers between light emitters and light detectors on the same side of a virtual plane to reduce the direct transmission of light from light emitters to light detectors without having been reflected by, or transmitted through, breast tissue. In an example, a device can further comprise opaque shielding and/or barriers between light emitters and light detectors on the same side of a virtual plane to reduce the direct transmission of light from light emitters to light detectors without having been reflected by, or transmitted through, breast tissue. In an example, opaque optical shielding between light emitters and detectors can be made from opaque elastomeric polymer material. In an example, opaque optical shielding between light emitters and detectors can be made from opaque conformable polymer material.

In an example, an optical layer can further comprise a plurality of elastic electroconductive pathways which are configured in a hub-and-spoke pattern. In an example, an optical layer can further comprise a plurality of elastic electroconductive pathways which are configured in a star burst pattern. In an example, an optical layer can further comprise a plurality of elastic electroconductive pathways which are configured in a spiral pattern.

In an example, electroconductive pathways which provide power to light emitters and detectors can be embroidered onto fabric. In an example, electromagnetic energy can be transmitted to light emitters through an undulating wire, conductive thread, or conductive yarn. In an example, flexible electroconductive pathways on an optical layer can be made from an elastomeric polymer (such as PDMS) which has been impregnated with carbon nanotubes. In an example, an optical layer can further comprise undulating (e.g. sinusoidal or zigzag) wires which are in electrical communication with light emitters and light detectors. In an example, flexible electroconductive pathways on an optical layer can be made from an elastomeric polymer (such as PDMS) which has been impregnated with conductive metal particles.

In an example, an optical layer can further comprise a plurality of elastic electroconductive pathways which are configured in a helical or half-helical pattern. In an example, an optical layer can further comprise a plurality of elastic electroconductive pathways which are configured in a row-and-column pattern. In an example, an optical layer can further comprise a plurality of elastic electroconductive pathways which are configured in an orthogonal matrix (e.g. quadrilateral grid or mesh).

In an example, electroconductive pathways in this device can be made with a combination of poly(3,4-ethylenedioxythiophene) polystyrene sulfonate (PEDOT:PSS) and carbon nanotubes. In an example, electroconductive pathways in this device can be made with a flexible and/or elastomeric material. In an example, electroconductive pathways in this device can be made with polydimethylsiloxane (PDMS) which has been doped, impregnated, and/or coated with electroconductive material. In an example, electroconductive pathways in this device can be made with a silicone-based polymer which has been doped, impregnated, and/or coated with electroconductive material. In an example, electroconductive pathways in this device can be made with polyethylene terephthalate (PET). In an example, electroconductive pathways in this device can be made with poly(3,4-ethylenedioxythiophene) polystyrene sulfonate (PEDOT:PSS).

In an example, an expandable layer can comprise a plurality of expandable components and/or sections. In an example, an expandable layer can comprise a plurality of inflatable (e.g. inflatable and/or pneumatic) chambers and/or sections. In an example, an expandable layer can comprise a plurality of chambers which are expanded by being filled with a gas (e.g. air). In an example, an expandable layer can comprise a plurality of inflatable chambers which are can be individually and selectively inflated y different degrees, by different amounts, to different sizes, and/or to different internal air pressures. In an example, an expandable layer can comprise a plurality of inflatable chambers which can be individually and selectively inflated so that they are inflated by different degrees, by different amounts, to different sizes, and/or to different internal pressures, thereby exerting different levels of pressure on different portions of the breast.

In an example, an expandable layer can comprise a plurality of inflatable chambers which can be individually and selectively inflated so that they are inflated by different degrees, by different amounts, to different sizes, and/or to different internal pressures, thereby compressing different portions of the breast by different extents. In an example, an expandable layer can comprise a plurality of inflatable chambers which can be individually and selectively inflated so that they compress wider portions of the breast to a greater degree than narrower portions of the breast, enabling better (e.g. more uniform) transmission of light through the breast.

In an example, an expandable layer can comprise a plurality of hydraulic chambers and/or sections. In an example, an expandable layer can comprise a plurality of hydraulic chambers which are expanded by being filled with a liquid. In an example, an expandable layer can comprise a plurality of hydraulic chambers which are can be individually and selectively expanded by different degrees, by different amounts, to different sizes, and/or to different internal air pressures. In an example, an expandable layer can comprise a plurality of hydraulic chambers which can be individually and selectively expanded so that they are expanded by different degrees, by different amounts, to different sizes, and/or to different internal pressures, thereby exerting different levels of pressure on different portions of the breast.

In an example, an expandable layer can comprise a plurality of hydraulic chambers which can be individually and selectively expanded so that they are expanded by different degrees, by different amounts, to different sizes, and/or to different internal pressures, thereby compressing different portions of the breast by different extents. In an example, an expandable layer can comprise a plurality of hydraulic chambers which can be individually and selectively expanded so that they compress wider portions of the breast to a greater degree than narrower portions of the breast, enabling better (e.g. more uniform) transmission of light through the breast.

In an example, an expandable component can be expanded by inflation with a gas (e.g. air). In an example, an expandable component can be an inflatable chamber with a flexible surface which is impermeable to air. In an example, an expandable component can be expanded by a pneumatic mechanism. In an example, an expandable component can be an air-tight compartment or pocket. In an example, an expandable component can be a balloon or inflatable bladder. In an example, an expandable component can be expanded by being filled with a liquid (e.g. saline solution). In an example, an expandable component can be expanded by liquid pressure. In an example, an expandable component can be a liquid-filled chamber or bladder with a flexible surface which is impermeable to liquid. In an example, an expandable component can be expanded by a hydraulic mechanism.

In an example, an expandable component can be expanded by filling it with a gas (such as air), a liquid (such as water), or a gel. In an example, the device can further comprise an air pump or liquid pump which is integrated into a bra. In another example, a pump can be separate from a wearable device, but connected to the device in order to expand the expandable components (e.g. via one or more tubes). In an example, a device can further comprise a data processor which controls the operation of an air pump or liquid pump. In an example, an expandable component can be expanded by a pneumatic mechanism. In an example, an expandable component can be expanded by pumping water into it. In an example, an expandable component can be expanded by a hydraulic mechanism.

In an example, a device can further comprise a liquid pump on the back strap of the bra, wherein the liquid pump is manually operated to pump liquid into one or more expandable components to compress a breast and improve the fit of the device to the contour of the breast. In an example, a device can include an air pump on the back strap of the bra, wherein the air pump is manually operated to pump air into one or more expandable components to compress a breast and improve the fit of the device to the contour of the breast. In an example, a pump can be manually operated by a person who presses the pump with their hand. In an example, a pump can be automatically operated by an impellor which is rotated by an electromagnetic motor.

In an example, this device can further comprise of tubes or channels which conduct a flowable substance (e.g. a gas or liquid) into a plurality of expandable components. In an example, there can be a separate air tube or channel for each expandable component. In an example, each expandable component can be in fluid communication with a pump through a separate fluid tube or channel between the pump and the expandable component. In an example, a smart bra can have ports and/or valves which connect internal air tubes through the body of the bra to external air tubes from a separate air pump. In an example, air tubes from the air pump can be connected to the bra via the ports and/or valves for expanding components within the cups of the bra. Alternatively, the back strap of the bra can include a built-in air pump which pumps air into expanding components when pressed repeatedly.

In an example, expandable components and/or sections which are closer to the center of an expandable layer can be expanded to a greater extent than components and/or sections which are farther from the center of the expandable layer. In an example, expandable components and/or sections which are closer to the center of the device can be expanded to a greater extent than components and/or sections which are farther from the center of the device. This can compress wider portions of the breast more than narrower portions of the breast, thereby reducing variation in the transmission distances of light traveling through breast tissue from light emitters to light detectors. In an example, a breast can be compressed into a flatter configuration (for better analysis by transmitted light) by individual and selectively expanding central expandable components and/or sections more than non-central expandable components and/or sections.

In an example, expansion of expandable components can compress a breast which is between them. This can enable more accurate optical scanning of breast tissue because light rays travel a shorter distance through tissue, with less scattering. In an example, a portion of a breast between two expandable components can have a first width when a device is in a first (unexpanded) configuration and a second width when the device is in a second (expanded) configuration, wherein the second width is less than the first width. In an example, a portion of the breast between the expandable components can be flatter in the second configuration than in the first configuration of the device.

In an example, expandable components (e.g. inflatable chambers) can be manually expanded by air pumped from a manually-activated air pump. This provides the person wearing the device with direct control over the pressure exerted by the expandable layer on the breast, which can be useful for avoiding undue (potentially painful) breast compression. In another example, expandable components can be automatically expanded, such as by an automatically-activated air pump. In an example, is expansion is automatic, then the device can further comprise one or more pressure sensors which monitor compression pressure on the breast and regulate pressure levels to avoid undue (potentially painful) breast compression.

In an example, this device can further comprise a compression-monitoring mechanism to monitor the amount of breast compression from expansion of the expandable level to ensure sufficient compression to get good analysis of breast tissue from transmission of light through the breast, but not so much compression than it causes pain, circulation problems, or other adverse effects. In an example, a compression-monitoring mechanism can be an optical mechanism. In an example, a compression-monitoring mechanism can comprise monitoring of the amount of light which is transmitted through the breast, especially the widest portion of the breast. Alternatively, a compression-monitoring mechanism can comprise one or more pressure sensors and/or strain sensor.

In an example, a device can identify the optimal compression profile for a specific breast (e.g. customized for the right or left breast of a specific person), wherein this compression profile includes expansion parameters for expandable components in the device. In an example, optimal inflation parameters for expandable components can be identified for a specific breast based on optical transmission results and/or pressure results. In an example, a device can be custom-fitted to a specific breast. In an example, a device can be custom-fitted by recording the optimal expansion parameters (e.g. found by trial and error) for expandable components in the device when the device is first fitted to a breast and then replicating those optimal expansion parameters when the device is again worn on the same breast. Custom-fitting the device to a specific breast can enhance the quality of optical sensing, achieve results more quickly, and avoid compression-related pain.

In an example, the amount of light passing through breast tissue between light emitters and light detectors can be monitored during expansion of the expandable components. In an example, expandable components can be expanded until the breast is sufficiently compressed that light received by light detectors reaches a target level of light transmission intensity or resolution. In an example, expandable components can be expanded until either a target level of light transmission is achieved or the person wearing the device indicates discomfort. In an example, expandable components can be expanded until a minimum target level and/or percentage of light emitted from light emitters is received by light detectors after passing through breast tissue. In an example, the expandable components are expanded until the amount of light scattering is reduced to a target level and/or percentage or the person wearing the device indicates discomfort.

In an example, this device can further comprise pressure sensors. In an example, these pressure sensors can measure the amount of pressure applied by the device to breast tissue to help avoid undue breast compression and discomfort. In an example, expansion of expandable components can be adjusted to try to achieve breast compression without undue pressure. In an example, expansion of one or more expandable components can be adjusted and/or controlled to achieve a desired width of the breast without undue pressure. In an example, expansion can be automatically stopped if a maximum target pressure is reached.

In an example, expansion of individual expandable components in an expandable layer can be individually and selectively controlled. In an example, expandable components in the expandable layer can be expanded to different extents, to different sizes, and/or to different internal pressure levels. In an example, expandable components which are closer to the center of the device can be expanded more than expandable components which are farther from the center of the device. In an example, expandable components in the expandable layer may be expanded to different extents so as to provide uniform pressure across the surface of a breast. In an example, expandable components in the expandable layer may be expanded to different extents so as to custom fit the device to breasts of different sizes and shapes.

In an example, one side of an expandable component can be more elastic (e.g. more elastic, more flexible, and/or less rigid) than other sides of the expandable component. In an example, having one side be more elastic can help to direct expansion of the expandable component toward the surface of a breast. In an example, a side of an expandable component which faces toward a breast can be more elastic than a side of the component which faces away from the breast. In an example, the side of an expandable component which faces toward an oblique virtual plane (spanning the upper out and lower inner quadrants) can be more elastic than the side of the component which faces away from that plane.

In an example, expansion of first and second expandable components in a device can be selectively and individually controlled. In an example, differential expansion of different components can help to compress a portion of a breast into a shape with more uniform width (e.g. flatter) for better scanning. In an example, a first expandable component can be expanded by a greater percentage than a second expandable component. In an example, a first expandable component can be expanded to a different size than a second expandable component.

In an example, the side and/or surface of an expandable component which faces toward the center of the breast can be more elastic than the side of the component which faces away from the center of the breast. In an example, the side and/or surface of an expandable component which faces away from the perimeter of a cup can be more elastic than the side of the component which faces toward the perimeter of the cup. In an example, the side and/or surface of an expandable component which faces away from the perimeter of a cup can have a lower Young's modulus than the side of the component which faces toward the perimeter of the cup. In an example, the side and/or surface of an expandable component which faces toward the center of the breast can have a lower Young's modulus than the side of the component which faces away from the center of the breast.

In an example, one side of an expandable component can be thinner than other sides of the expandable component. In an example, having one side be thinner can help to direct expansion of the expandable component toward the surface of a breast. In an example, a side of an expandable component which faces toward a breast can be thinner than a side of the component which faces away from the breast. In an example, the side of an expandable component which faces toward an oblique virtual plane (spanning the upper out and lower inner quadrants) can be thinner than the side of the component which faces away from that plane.

In an example, expansion of individual expandable components in an expandable layer can be individually and selectively controlled. In an example, expandable components in the expandable layer can be expanded at different times and/or in a sequential manner. In an example, expansion of expandable components in a sequential manner can create peristaltic motion which causes a wave of compression of breast tissue. In an example, a compressive wave can achieve more-uniform post-compression tissue width and/or help to avoid pain during breast compression. In an example, expandable components which are closer to the center of the device can be expanded before expandable components which are farther from the center of the device. In an example, the sequence of expansion of expandable components can be adjusted and/or programmed to achieve different post-compression tissue width outcomes.

In an example, different expandable components can be expanded at different times. In an example, the timing of expansion of two or more expandable components can be individually and selectively controlled. In an example, expandable components which are closer to the center of a device can be expanded before expandable components which are farther from the center of the device. In an example, expandable components which are closer to wider portions of a breast can be expanded before expandable components which are farther from wider portions of the breast.

In an example, expandable components can have keystone and/or trapezoidal shapes when they are expanded. In an example, expandable components can expand predominately along (radial) vectors toward the virtual plane. In an example, one part of an expandable layer which is on one side of the virtual plane can have a plurality of expandable components. In an example, one part of an expandable layer which is on one side of the virtual plane can have at least one expandable component in each quadrant spanned by the part.

In an example, a first part of an expandable layer which is on a first side of an oblique virtual plane can have at least one expandable component in each of: a portion of the expandable layer over the upper outer quadrant of a breast; a portion of the expandable layer over the lower outer quadrant of the breast; and a portion of the expandable layer over the lower inner quadrant of the breast. In an example, a second part of the expandable layer which is on a second (opposite) side of an oblique virtual plane can have at least one expandable component in each of: a portion of the expandable layer over the upper outer quadrant of a breast; a portion of the expandable layer over the upper inner quadrant of the breast; and a portion of the expandable layer over the lower inner quadrant of the breast.

In an example, a first part of an expandable layer which is on a first side of an oblique virtual plane can comprise: at least one expandable component on a portion of the expandable layer over the upper outer quadrant of a breast; at least two expandable components on a portion of the expandable layer over the lower outer quadrant of the breast; and at least one expandable component on the portion of the expandable layer over the lower inner quadrant of the breast. In an example, a second part of the expandable layer which is on a second (e.g. opposite) side of an oblique virtual plane can comprise: at least one expandable component on a portion of the expandable layer over the upper outer quadrant of a breast; at least two expandable components on a portion of the expandable layer over the upper inner quadrant of the breast; and at least one expandable component on the portion of the expandable layer over the lower inner quadrant of the breast.

In an example, a first part of an expandable layer which is on a first side of an oblique virtual plane can have more expandable components over the lower outer quadrant of the breast than over the lower inner quadrant of the breast. In an example, a second part of the expandable layer which is on a second (opposite) side of the oblique virtual plane can have more expandable components over the upper inner quadrant of the breast than over the lower inner quadrant of the breast.

In an example, an expandable component can have an arcuate shape. In an example, an expandable component can have a conic-section shape. In an example, an expandable component can be shaped like a section of a circle, ring, or torus. In an example, an expandable component can be shaped like a half of a circle, ring, or torus. In an example, an expandable component can be shaped like a quarter of a circle, ring, or torus. In an example, an expandable component can have a crescent or banana shape. In an example, an expandable component can have a keystone or trapezoidal shape. In an example, an expandable component can have a toroidal or doughnut shape.

In an example, an expandable component can have a pleated and/or folded shape like an accordion or bellows. In an example, an expandable component can have a disk shape in a first configuration and an ellipsoidal shape in an expanded second configuration. In an example, an expandable component can have a disk shape in a first configuration and a cylindrical shape in an expanded second configuration. In an example, an expandable component can have a shape which is selected from the group consisting of: pancake, disk, ellipsoidal, oblong, oval, toroidal, hemispherical, and spherical.

In an example, there can be different numbers, sizes, shapes, and/or elasticities of expandable components in different quadrants of a device. In an example, expandable components in portions of a device which cover the lower outer and upper inner quadrants of the breast can be larger than expandable components in other portions of the device. In an example, expandable components in portions of a device which cover the lower outer and upper inner quadrants of the breast can have different shapes than expandable components in other portions of the device. In an example, expandable components in portions of a device which cover the lower outer and upper inner quadrants of the breast can be less triangular and/or more trapezoidal than expandable components in other portions of the device. In an example, expandable components in portions of a device which cover the lower outer and upper inner quadrants of the breast can be more elastic and/or stretchable than expandable components in other portions of the device.

In an example, an expandable layer can be substantially symmetric with respect to reflection across a virtual plane. In an example, a first part of an expandable layer on a first side of a virtual plane can have the same number of expandable components as a second part of the expandable layer on a second (e.g. opposite) side of the virtual plane. In an example, expandable components in a first part of an expandable layer on a first side of a virtual plane can have substantially the same sizes and/or shapes as expandable components in a second part of the expandable layer on a second (e.g. opposite) side of the virtual plane.

In an example, an expandable layer can be asymmetric with respect to reflection across a virtual plane. In an example, a first part of an expandable layer on a first side of a virtual plane can have more expandable components than a second part of the expandable layer on a second (e.g. opposite) side of the virtual plane. In an example, expandable components in a first part of an expandable layer on a first side of a virtual plane can have different sizes and/or shapes than expandable components in a second part of the expandable layer on a second (e.g. opposite) side of the virtual plane.

In an example, a first expandable component can be in a first (e.g. lower-left) half of a device which is on a first side of an oblique virtual plane which intersects the device and a second expandable component can be in a second (e.g. upper-right) half of a device which is on a second side of the plane. In an example, first and second expandable components can be located to the lower left and to the upper right, respectively, of a 45-degree diagonal anterior-to-posterior virtual plane which intersects the device.

In an example, a first expandable component can be in a first (e.g. right) half of a device which is on a first side of a vertical virtual plane which intersects the device and a second expandable component can be in a second (e.g. left) half of a device which is on a second side of the plane. In an example, first and second expandable components can be located to the left and to the right, respectively, of a vertical anterior-to-posterior virtual plane which intersects the device. In an example, a first expandable component can be in a first (e.g. upper) half of a device which is on a first side of a horizontal virtual plane which intersects the device and a second expandable component can be in a second (e.g. lower) half of a device which is on a second side of the plane. In an example, first and second expandable components can be located below and above, respectively, a horizontal anterior-to-posterior virtual plane which intersects the device.

In an example, a first expandable component can be in a first half of a device which is on a first side of a virtual plane which intersects the device and no closer than ¼ inch from the plane. In an example, a first expandable component can be in a first half of a device which is on a first side of a virtual plane which intersects the device and no closer than ½ inch from the plane. In an example, a first expandable component can be in a first half of a device which is on a first side of a virtual plane which intersects the device and no closer than 1 inch from the plane. In an example, a second expandable component can be in a second half of a device which is on a second side of the plane and no closer than ¼ inch from the plane. In an example, a second expandable component can be in a second half of a device which is on a second side of the plane and no closer than ½ inch from the plane. In an example, a second expandable component can be in a second half of a device which is on a second side of the plane and no closer than 1 inch from the plane.

In an example a device can comprise a first expandable component on (or in) the right side of the concave interior of a cup and a second expandable component on (or in) the left side of the concave interior of the cup. In an example, a device can comprise a first expandable component on (or in) the upper half of the interior of a cup and a second expandable component on (or in) the lower half of the interior of the cup. In an example, a device can comprise a first expandable component on (or in) the upper right quadrant (from a frontal corona view) of a cup for a right-side breast and a second expandable component on (or in) the lower left quadrant (from a frontal corona view) of the interior of the cup. In an example, a device can comprise a first expandable component on (or in) the upper left quadrant (from a frontal corona view) of a cup for a left-side breast and a second expandable component on (or in) the lower right quadrant (from a frontal corona view) of the interior of the cup.

In an example, an expandable layer can span all of the interior concavity of the device. In an example, an expandable layer can span between 50% and 75% of the interior concavity of the device. In an example, an expandable layer can span between 60% and 85% of the interior concavity of the device. In an example, an expandable layer can span the entire perimeter of the device. In an example, an expandable layer can span between 50% and 75% of the perimeter of the device. In an example, an expandable layer can span between 60% and 85% of the perimeter of the device.

In an example, a structural layer can be less flexible, less elastic, and/or more rigid than other layers of the device. In an example, a structural layer can restrict outward expansion of expandable components in the expandable layer so that expansion of these components is directly primarily inward toward the breast. In an example, a structural layer can restrict outward expansion of expandable components in the expandable layer so that expansion of these components is directly primarily inward to compress the breast. In an example, the structural layer can be the exterior layer of the device, facing away from the breast. In an example, the structural layer can be the exterior layer of the device, facing away from concave interior of the breast.

In an example, the flexibility, elasticity, and/or rigidity of the structural layer can be non-uniform. In an example, the base of the structural layer which is closest to the chest wall can be less flexible, less elastic, and/or more rigid than the rest of the structural layer. In an example, the center of the structural layer can be more flexible, more elastic, and/or less rigid than the perimeter of the structural layer. In an example, areas of the structural layer which are closer to the virtual plane separating other layers can be more flexible, more elastic, and/or less rigid than other areas of the structural layer in order to allow the breast to expand along one axis (e.g. dorsal to ventral) when it is compressed along another axis (e.g. oblique).

In an example, there can be variation in the elasticity (e.g. Young's modulus), stretchability, and/or rigidity of different portions of an (outer) structural layer of this device. In an example, a structural layer can further comprise relatively inelastic components (e.g. wires or plastic strips) which are embedded in some portions of the layer. In an example, having some portions of a structural layer provide more resistance to outward expansion than other portions can help to direct expansion of components in an expandable layer inward toward a breast so as to selectively compress the breast into a flatter configuration for better optical scanning.

In an example, a central portion of a device can be more elastic (e.g. lower Young's modulus) and/or less rigid than other portions of the device. In an example, a portion of a device which is closer to the apex of a cup can be more elastic and/or less rigid than other portions of the device. In an example, portions of a device which are closer to an oblique virtual plane which spans between the upper outer quadrant and the lower inner quadrant of the breast can be more elastic (e.g. lower Young's modulus) and/or more rigid than other portions of the device.

In an example, perimeter portions of a device can be more elastic and/or less rigid than other portions of the device. In an example, a central portion of a device can be less elastic (e.g. higher Young's modulus) and/or more rigid than other portions of the device. In an example, a portion of a device which is closer to the apex of a cup can be less elastic and/or more rigid than other portions of the device.

In an example, portions of a device which are closer to an oblique virtual plane which spans between the upper outer quadrant and the lower inner quadrant of the breast can be less elastic (e.g. higher Young's modulus) and/or more rigid than other portions of the device. In an example, perimeter portions of a device can be less elastic and/or more rigid than other portions of the device. In an example, perimeter portions of a cup can be less elastic and/or more rigid. In an example, the perimeter of device which is closest to the chest wall can be less elastic (e.g. higher Young's modulus) and/or more rigid than other portions of the cup.

In an example, a structural layer can have a concave shape. In an example, a structural layer can have a substantially planar shape before being worn on a breast and change into a convex shape as it is worn on a breast. In an example, a cross-section of a structural layer can have a teardrop cross-sectional shape. In an example, the apex of this teardrop shape can encompass the Auxiliary Tail of Spence. In an example, a cross-section of a structural layer can have a shape whose two-dimensional parametric equation is X=cos(T) and Y=[sin(T)][sin M(T/2)].

In an example, portions of a structural layer which cover the upper inner, lower inner, and lower outer quadrants of the breast can have quarter-circle (e.g. quarter pie slice) cross-sectional perimeters. In an example, the portion of the device which covers the upper outer quadrant and the Auxiliary Tail of Spence can have a quadrilateral cross-sectional perimeter. In another example, a cross-section of a structural layer can have an elliptical or oval shape. In an example, a cross-section of a structural layer can have a paisley shape.

In an example, an (outer) structural layer of a device can be concave when the device is in an expanded configuration. In an example, an (outer) structural layer of a device can have a shape which is selected from the group consisting of: hemisphere, half of an oblate spheroid, half of an ellipsoid, half of a 3D teardrop shape, and conic section. In an example, the perimeter of the portion of the device which is closest to the chest wall can have a teardrop shape, wherein the vertex of the teardrop is aligned with the Auxiliary Tail of Spence.

In an example, one or more layers of this device can be rotated. In an example, one or more layers of this device can be rotated to change the orientation of the virtual plane and to compress the breast from different angles. In an example, one or more layers of this device can be rotated to optically analyze the breast from different angles and/or provide more in-depth analysis of different breast quadrants. In an example, this device can further comprise an inclinometer in order to register (e.g. align) the device relative to a vertical plane. In an example, a device can be rotated and/or shifted to align the device with anatomical locations and/or features of a breast in order to register and/or align optical scans taken at different times (e.g. when the person wears the device at different times).

In an example, this device can further comprise one or more marks, indicators, openings, or sensors which help to register (e.g. identify) the location of the device relative to a selected location, anatomical feature, or orientation of a breast. In an example, this device can further comprise one or more marks, indicators, or sensors which help a person to place the device on the same location on a breast and/or in the same orientation at different times during repeated uses (e.g. when the device is worn at different times). In an example, a device can have one or more marks which align with specific locations on a bra, thereby achieving a desired location and/or orientation relative to a breast. In an example, a device can further comprise an inclinometer which enables positioning the device in the same orientation relative to a vertical plane in repeated uses (e.g. wearing the device at different times).

In an example, one or more portions of the device can be rotated relative to other portions of the device in order to adjust the portions of the breast which are compressed and/or optically scanned. In an example, an optical layer of the device can be rotated relative to the structural layer of the device in order to adjust the portions of the breast which are compressed and/or optically scanned. In an example, an expandable layer of the device can be rotated relative to the structural layer of the device in order to adjust the portions of the breast which are compressed and/or optically scanned. In an example, one or more portions of the device can be rotated relative to other portions of the device in order to change the quadrants of the breast which are most compressed and/or optically scanned. In an example, an optical layer of the device can be rotated relative to the structural layer of the device in order to change the quadrants of the breast which are most compressed and/or optically scanned.

In an example, a device which is inserted between a bra cup and a breast can be rotated and/or flipped between being used on a right breast and being used on a left breast. Such rotation and/or flipping can enable the device to be designed with to provide more accurate scanning of the upper outer quadrant and/or Auxiliary Tail of Spence because this area has a higher probability of developing malignant tissue. In an example, the entire device can be rotated and/or flipped from right breast use to left breast use. Alternatively, only a portion of the device may be rotated and/or flipped. In an example, only one or more internal layers (e.g. the optical layer and the air-gap-reducing layer) may be rotated and/or flipped.

In an example, a perimeter of the portion of the device which contacts the chest wall can have a tear-drop shape, wherein the apex and/or vertex of this perimeter is aligned with the Auxiliary Tail of Spence. In an example, the perimeter of the portion of the device which contacts the chest wall can have a tear-drop shape, wherein the device is rotated between right breast and left breast applications so that the apex and/or vertex of this perimeter is aligned with the Auxiliary Tail of Spence in both applications. In an example, the device can have marks or openings which are aligned anatomical features on a person's body to register the location of the device and ensure placement alignment during repeated uses at different times. This can be useful for tracking possible changes in specific locations of breast tissue over time.

In an example, a device can be rotated for use in different orientations on the same breast. In an example, a device can be rotated so that light emitters and light detectors are on either side of an oblique virtual plane which spans between the upper outer quadrant of the breast and the lower inner quadrant of the breast. In an example, a device can then be rotated for scanning from a different angle so that the same light emitters and light detectors are on either side of a vertical plane, with the upper and lower outer quadrants on one side of the plane and the upper and lower inner quadrants on the other side of the plane. In an example, a device can then be rotated for scanning from a different angle so that the same light emitters and light detectors are on either side of a horizontal plane, with the outer and inner upper quadrants on one side of the plane and the outer and lower quadrants on the other side of the plane.

In an example a virtual plane can be oriented to span between the upper outer quadrant (and the Auxiliary Tail of Space) to the lower inner quadrant so that light emitters can be as close as possible to the chest wall near the upper outer quadrant when the breast is compressed. This is desirable for optical identification of abnormal breast tissue because breast cancer is most prevalent in this quadrant. In an example, an oblique virtual plane can bisect the upper outer quadrant and bisect the lower inner quadrant. In an example, an oblique virtual plane can diagonally bisect the upper outer quadrant and diagonally bisect the lower inner quadrant.

In an alternative example, a wearable device for optical scanning of breast tissue can comprise only three layers: an optical layer, an expandable layer, and a structural layer. In an alternative example, a wearable device for optical scanning of breast tissue can comprise only three layers: an air-gap-reducing layer, an optical layer, and a structural layer. In an example, a first portion of a device can comprise all four layers (e.g. air-gap-reducing layer, optical layer, expandable layer, and structural layer) and a second portion of a device can comprise only three of these layers.

In an example, a device can comprise only three layers: an inner air-gap-reducing layer (closest to the surface of the breast), an optical layer, and an outer structural layer (farthest from the surface of the breast). In an example, the optical layer can comprise light emitters and light detectors. In an example, the inner air-gap-reducing layer can be sufficiently conformable, flexible, and thick that it can greatly reduce (or even eliminate) air gaps between the optical layer and the surface of the breast, even for breasts with different sizes and shapes. In an example, the inner air-gap-reducing layer can comprise a conformable gel which is either transparent or has optical qualities which are similar to those of normal breast tissue.

In an example, a device can comprise only three layers: an inner optical layer (closest to the surface of the breast), and middle expandable layer, and an outer structural layer (farthest from the surface of the breast). In an example, the inner optical layer can comprise light emitters and light detectors which are in direct contact with the surface of a breast. In an example, the expandable layer can be sufficiently controllable and flexible that it can greatly reduce (or even eliminate) air gaps between the optical layer and the surface of the breast, even for breasts with different sizes and shapes. In an example, the expandable layer can have a sufficiently large number of individually-controllable expandable components that is can cause the optical layer to conform to the shape of a particular breast with a specific size and shape, thereby reducing (or even eliminating) air gaps between the optical layer and the surface of the breast.

In an example, light which has been transmitted through and/or reflected from breast tissue can be analyzed to detect and/or image levels, concentrations, and/or locations of deoxyhemoglobin in breast tissue. In an example, light which has been transmitted through and/or reflected from breast tissue can be analyzed to detect and/or image the locations, sizes, and/or configurations of extracellular matrix structures in breast tissue. In an example, light which has been transmitted through and/or reflected from breast tissue can be analyzed to detect and/or image levels, concentrations, and/or locations of oxygenated hemoglobin in breast tissue.

In an example, light which has been transmitted through and/or reflected from breast tissue can be analyzed to detect and/or image the locations, sizes, and/or configurations of vasculature sprouting in breast tissue. In an example, light which has been transmitted through and/or reflected from breast tissue can be analyzed to detect and/or image levels, concentrations, and/or locations of water in breast tissue. In an example, light which has been transmitted through and/or reflected from breast tissue can be analyzed to detect and/or image levels, concentrations, and/or locations of collagen in breast tissue.

In an example, light which has been transmitted through and/or reflected from breast tissue can be analyzed to detect and/or image levels, concentrations, and/or locations of hemoglobin in breast tissue. In an example, light which has been transmitted through and/or reflected from breast tissue can be analyzed to detect and/or image levels, concentrations, and/or locations of lipids in breast tissue. In an example, light which has been transmitted through and/or reflected from breast tissue can be analyzed to detect and/or image levels, concentrations, and/or locations of oxygen in breast tissue. In an example, light which has been transmitted through and/or reflected from breast tissue can be analyzed to detect and/or image the locations and configurations of lymphatics in breast tissue.

In an example, light which has been transmitted through and/or reflected from breast tissue and received by light detectors can be analyzed to detect and/or image abnormal breast tissue using one or more methods selected from the group consisting of: Time Reversal Optical Tomography (TROT), changes in the frequency spectrum of light transmitted through a breast, Diffuse Optical Imaging (DOI), Diffuse Optical Tomography (DOT), spectroscopic analysis, analysis of absorption and/or scattering of light transmitted through a breast, Near-Infrared Spectroscopy (NIRS), functional Near-Infrared Spectroscopy (fNIRS), changes in the intensity or amplitude of light transmitted through a breast, changes in the phase of light transmitted through a breast, Diffuse Correlation Spectroscopy (DCS), Carlavian Curve Analysis (CCA), machine learning, neural network analysis, broadband spectroscopy, and/or changes in the spectral distribution of light transmitted through a breast.

In an example, this device can use Diffuse Optical Imaging (DOI) to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue. In an example, this device can use Diffuse Optical Tomography (DOT) to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue. In an example, this device can use time of flight DOT to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue.

In an example, this device can use Near-Infrared Spectroscopy (NIRS) to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue. In an example, this device can use Raman scattering to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue. In an example, the results from optically-scanning right and left breasts can be compared and/or contrasted to each other to help detect abnormal breast tissue.

In an example, this device can be used for one or more optical scanning methods selected from the group consisting of: Diffuse Optical Imaging (DOI), Diffuse Optical Spectroscopic Imaging (DOSI), Diffuse Optical Spectroscopy (DOS), Diffuse Optical Tomography (DOT), Frequency-Domain Photon Migration (FDPM), Functional Near-Infrared Spectroscopy (fNIRS), Near-Infrared Spectroscopy (NIRS), Raman spectroscopy, Reflectance Diffuse Optical Tomography (RDOT), Transillumination Imaging (TI), and/or Transmittance Diffuse Optical Tomography (TDOT). In an example, this device can be used for continuous wave (CW) optical analysis. In an example, this device can be used frequency domain (FD) optical analysis. In an example, this device can be used for time domain (TD) optical analysis.

In an example, changes in the direction of light caused by its transmission through breast tissue can be analyzed to create an image of the breast tissue. In an example, changes in the direction of light caused by its transmission through breast tissue can be analyzed to identify the levels, concentrations, and/or locations of specific biological substances (e.g. markers associated with abnormal tissue) in the breast tissue. In an example, changes in the intensity of light caused by its transmission through breast tissue can be analyzed to create an image of the breast tissue. In an example, changes in the intensity of light caused by its transmission through breast tissue can be analyzed to identify the levels, concentrations, and/or locations of specific biological substances (e.g. markers associated with abnormal tissue) in the breast tissue.

In an example, changes in the spectrum of light caused by its transmission through breast tissue can be analyzed to create an image of the breast tissue. In an example, changes in the spectrum of light caused by its transmission through breast tissue can be analyzed to identify the levels, concentrations, and/or locations of specific biological substances (e.g. markers associated with abnormal tissue) in the breast tissue. In an example, spectroscopic analysis of light which has been transmitted through breast tissue can be done to identify the levels, concentrations, and/or locations of specific biological substances (e.g. markers associated with abnormal tissue) in the breast tissue.

In an example, spectroscopic analysis of light which has been transmitted through and/or reflected from breast tissue can be done to identify the levels, concentrations, and/or locations of specific biological substances (e.g. markers associated with abnormal tissue) in the breast tissue. In an example, spectroscopic analysis of light which has been transmitted through and/or reflected from breast tissue can be done to identify the levels, concentrations, and/or locations of specific biological substances (e.g. markers associated with abnormal tissue) in the breast tissue. In an example, spectroscopic analysis of light which has been transmitted through and/or reflected from breast tissue can be done to identify the sizes, configurations, and/or locations of specific biological structures associated with abnormal tissue.

In an example, spectroscopic analysis of light which has been transmitted through and/or reflected from breast tissue can be done to identify the sizes, configurations, and/or locations of specific biological structures associated with abnormal tissue. In an example, spectroscopic analysis of light which has been transmitted through and/or reflected from breast tissue can be done to identify the sizes, configurations, and/or locations of specific biological structures associated with abnormal tissue. In an example, spectroscopic analysis of near-infrared light which has been transmitted through and/or reflected from breast tissue can be done to identify the levels, concentrations, and/or locations of specific biological substances (e.g. markers associated with abnormal tissue) in the breast tissue. In an example, spectroscopic analysis of near-infrared light which has been transmitted through and/or reflected from breast tissue can be done to identify the levels, concentrations, and/or locations of specific biological substances (e.g. markers associated with abnormal tissue) in the breast tissue.

In an example, spectroscopic analysis of near-infrared light which has been transmitted through and/or reflected from breast tissue can be done to identify the levels, concentrations, and/or locations of specific biological substances (e.g. markers associated with abnormal tissue) in the breast tissue. In an example, spectroscopic analysis of near-infrared light which has been transmitted through and/or reflected from breast tissue can be done to identify the sizes, configurations, and/or locations of specific biological structures associated with abnormal tissue. In an example, spectroscopic analysis of near-infrared light which has been transmitted through and/or reflected from breast tissue can be done to identify the sizes, configurations, and/or locations of specific biological structures associated with abnormal tissue. In an example, spectroscopic analysis of near-infrared light which has been transmitted through and/or reflected from breast tissue can be done to identify the sizes, configurations, and/or locations of specific biological structures associated with abnormal tissue.

In an example, changes in the direction of light caused by its reflection from breast tissue can be analyzed to create an image of the breast tissue. In an example, changes in the direction of light caused by its reflection from breast tissue can be analyzed to identify the levels, concentrations, and/or locations of specific biological substances (e.g. markers associated with abnormal tissue) in the breast tissue. In an example, changes in the intensity of light caused by its reflection from breast tissue can be analyzed to create an image of the breast tissue. In an example, changes in the intensity of light caused by its reflection from breast tissue can be analyzed to identify the levels, concentrations, and/or locations of specific biological substances (e.g. markers associated with abnormal tissue) in the breast tissue.

In an example, changes in the spectrum of light caused by its reflection from breast tissue can be analyzed to create an image of the breast tissue. In an example, changes in the spectrum of light caused by its reflection from breast tissue can be analyzed to identify the levels, concentrations, and/or locations of specific biological substances (e.g. markers associated with abnormal tissue) in the breast tissue. In an example, spectroscopic analysis of light which has been reflected from breast tissue can be done to identify the levels, concentrations, and/or locations of specific biological substances (e.g. markers associated with abnormal tissue) in the breast tissue.

In an example, spectroscopic analysis of light which has been reflected from breast tissue can be done to identify the levels, concentrations, and/or locations of specific biological substances (e.g. markers associated with abnormal tissue) in the breast tissue. In an example, spectroscopic analysis of light which has been reflected from breast tissue can be done to identify the levels, concentrations, and/or locations of specific biological substances (e.g. markers associated with abnormal tissue) in the breast tissue. In an example, spectroscopic analysis of light which has been reflected from breast tissue can be done to identify the sizes, configurations, and/or locations of specific biological structures associated with abnormal tissue.

In an example, spectroscopic analysis of light which has been reflected from breast tissue can be done to identify the sizes, configurations, and/or locations of specific biological structures associated with abnormal tissue. In an example, spectroscopic analysis of light which has been reflected from breast tissue can be done to identify the sizes, configurations, and/or locations of specific biological structures associated with abnormal tissue. In an example, spectroscopic analysis of near-infrared light which has been reflected from breast tissue can be done to identify the levels, concentrations, and/or locations of specific biological substances (e.g. markers associated with abnormal tissue) in the breast tissue. In an example, spectroscopic analysis of near-infrared light which has been reflected from breast tissue can be done to identify the levels, concentrations, and/or locations of specific biological substances (e.g. markers associated with abnormal tissue) in the breast tissue.

In an example, spectroscopic analysis of near-infrared light which has been reflected from breast tissue can be done to identify the levels, concentrations, and/or locations of specific biological substances (e.g. markers associated with abnormal tissue) in the breast tissue. In an example, spectroscopic analysis of near-infrared light which has been reflected from breast tissue can be done to identify the sizes, configurations, and/or locations of specific biological structures associated with abnormal tissue. In an example, spectroscopic analysis of near-infrared light which has been reflected from breast tissue can be done to identify the sizes, configurations, and/or locations of specific biological structures associated with abnormal tissue. In an example, spectroscopic analysis of near-infrared light which has been reflected from breast tissue can be done to identify the sizes, configurations, and/or locations of specific biological structures associated with abnormal tissue.

In an example, changes in the intensity and/or spectral distribution of light caused by transmission through breast tissue can be analyzed to create a (3D) image of breast tissue. In an example, changes in the intensity and/or spectral distribution of light caused by transmission through breast tissue can be analyzed to create a (3D) image which shows variation in breast tissue composition. In an example, changes in the intensity and/or spectral distribution of light caused by transmission through breast tissue can be analyzed to create a (3D) image which shows variation in breast tissue structure. In an example, changes in the intensity and/or spectral distribution of light caused by transmission through breast tissue can be analyzed to create a (3D) image which shows the locations, sizes, and shapes of abnormal breast tissue. In an example, changes in the intensity and/or spectral distribution of light caused by transmission through breast tissue can be analyzed to create a (3D) image which shows levels and/or concentrations of biological substances (e.g. markers) which are associated with abnormal tissue.

In an example, changes in the intensity (e.g. amplitude) of light emitted from light emitters and received by light detectors which are caused by passage of the light through breast tissue can be analyzed to detect abnormal breast tissue. In an example, changes in the intensity (e.g. amplitude) of light emitted from light emitters and received by light detectors which are caused by passage of the light through breast tissue can be analyzed to image breast tissue. In an example, changes in the intensity (e.g. amplitude) of light emitted from light emitters and received by light detectors which are caused by passage of the light through breast tissue can be analyzed to evaluate the size, shape, density, and/or location of abnormal breast tissue. In an example, changes in the intensity (e.g. amplitude) of light emitted from light emitters and received by light detectors which are caused by passage of the light through breast tissue can be analyzed to evaluate the molecular composition of breast tissue and detect abnormal breast tissue.

In an example, changes in the spectrum (e.g. spectral distribution) of light emitted from light emitters and received by light detectors which are caused by passage of the light through breast tissue can be analyzed to detect abnormal breast tissue. In an example, changes in the spectrum (e.g. spectral distribution) of light emitted from light emitters and received by light detectors which are caused by passage of the light through breast tissue can be analyzed to image breast tissue. In an example, changes in the spectrum (e.g. spectral distribution) of light emitted from light emitters and received by light detectors which are caused by passage of the light through breast tissue can be analyzed to evaluate the size, shape, density, and/or location of abnormal breast tissue. In an example, changes in the spectrum (e.g. spectral distribution) of light emitted from light emitters and received by light detectors which are caused by passage of the light through breast tissue can be analyzed to evaluate the molecular composition of breast tissue and detect abnormal breast tissue.

Although light energy is significantly diffused through the depth of breast tissue, joint three-dimensional analysis of light transmitted through multiple intersecting vectors between multiple pairs of light emitters and light detectors can provide parallel pathway data which increases the accuracy and locational precision of spectroscopic analysis in order to identify and locate abnormal tissue. Joint analysis of the intensity and spectral changes of light beams traveling through the breast tissue along different three-dimensional vectors can identify whether there is abnormal tissue within the breast and, if so, where the abnormal tissue is located. In an example, light which has been transmitted through breast tissue between different pairs of light emitters and light detectors (at different times) can be triangulated in order to identify the presence, composition, shape, size, and/or location of abnormal tissue.

In an example, this device can be worn for a short period of time (on a periodic basis, such as annually, monthly, weekly, or daily) in order to obtain a periodic longitudinal time series of optical of breast tissue for identification of changes in tissue composition. In an example, this device can be worn periodically in order to obtain a periodic longitudinal time series of optical scans of breast tissue. In an example, changes in tissue composition over time can be identified which could indicate abnormal tissue growth. In an example, results from more recent scans can be compared and/or contrasted with earlier scans to help detect growth of abnormal breast tissue.

In an example, this device can further comprise a power source. In an example, a power source can be a battery. In an example, a power source which powers light emitters and other components can be an integral part of the device. In an example, a power source can be located on a posterior portion of a smart bra. In an example, a power source can be located on the back strap of a smart bra.

In an example, this device can further comprise a data processor. In an example, this data processor can control the light emitters and light detectors. In an example, a device can further comprise a local data processor which is physically integrated into the device. In an example, a device can further comprise a local data processor which is an integral part of a smart bra. In an example, a device can further comprise a local data processor which is located on the posterior portion of a smart bra. In an example, a device can further comprise a local data processor which is located on the back strap of a smart bra. In an example, a device can further comprise a local data processer which is in electronic communication with a remote data processor.

In an example, a data processor can receive data from the light detectors. In an example, a device can be a component of a system which includes a local data processor, a local data transmitter (which are contiguous parts of the device) and a remote (non-contiguous) data processor which receives data from the data transmitter. In an example, a system can comprise a local data processor and data transmitter which are part of a smart bra and a remote data processor which receives data from the local data processor via the data transmitter. In an example, a system can comprise a smart bra which is in wireless communication with a cell phone, smart watch, smart glasses, tablet computer, laptop computer, or remote server.

In an example, analysis of changes in light intensity and/or spectral distribution caused by light transmission through and/or reflection from breast tissue can be analyzed in a remote data processor. In an example, data from light detectors can be transmitted to a separate and/or remote data processor for spectroscopic analysis to identify changes in breast tissue composition and/or to identify abnormal breast tissue. In an example, a remote data processor can be in another wearable device (e.g. a smart watch), a mobile device (e.g. a cell phone), or a remote server (e.g. in a healthcare provider's server and/or cloud storage). In an example, data from a wearable device can be wirelessly transmitted to a data processor in a different wearable device (e.g. a smart watch), a handheld device (e.g. a cell phone), or a remote server (e.g. in a healthcare provider's server and/or cloud storage).

In an example, a device (or a system of which a device is a part) can further comprise one or more other components selected from the group consisting of: power source (e.g. battery), flexible electroconductive wires and/or textile channels, data processor (local, remote, or both local and remote), wireless data transmitter, wireless data receiver, pressure sensors, motion sensors (e.g. accelerometer and gyroscope), inclinometer, mirrors (e.g. micromirror array), pump and/or impellor (e.g. air or liquid pump or impellor), tubes (e.g. air or liquid conducting tubes), air or liquid reservoir, and electromagnetic actuator. In an example, if this device is embodied in a bra, then one or more of these components can be located on the back strap of the bra. In an example, one or more of these components can be temporarily removed from a device so that the rest of the device can be washed.

In an example, a multi-layer device for optical detection of breast cancer can comprise: an air-gap-reducing layer which is configured to be worn on the surface of a person's breast, wherein the air-gap-reducing layer is transparent or has the same optical characteristics as normal breast tissue, and wherein a first part of the air-gap-reducing layer is on a first side of a virtual plane and a second part of the air-gap-reducing layer is on a second side of the virtual plane; an optical layer with a plurality of light emitters and light detectors, wherein a first part of the optical layer is on the first side of the virtual plane and a second part of the optical layer is on the second side of the virtual plane; an expandable layer with a plurality of expandable components, wherein a first part of the expandable layer is on the first side of the virtual plane and a second part of the expandable layer is on the second side of the virtual plane; and a structural layer which reduces expansion of the expandable components away from the breast; wherein the optical layer is between the air-gap-reducing layer and the expandable layer, wherein the expandable layer is between the optical layer and the structural layer, wherein the device has an unexpanded configuration in which the expandable components are not expanded, wherein the device has an expanded configuration in which the expandable components are expanded, wherein there is a first average distance between the first part of the optical layer and the second part of the optical layer when the device is in the unexpanded configuration, wherein there is a second average distance between the first part of the optical layer and the second part of the optical layer when the device is in the expanded configuration, and wherein the second average distance is less than the first average distance.

In an example, the device can be a bra. In an example, the device can be inserted into a bra cup. In an example, the device can be worn between a bra cup and a breast. In an example, the device can be an adhesive patch, sticker, or bandage. In an example, the device can have a teardrop-shaped perimeter. In an example, the teardrop shape of the perimeter can have an apex and/or vertex which is placed over the Auxiliary Tail of Spence. In an example, the virtual plane can be an oblique virtual plane which spans from the upper outer quadrant to the lower inner quadrant of the breast.

In an example, there can be light emitters on the first side of the virtual plane and light detectors on the second side of the virtual plane. In an example, light from the light emitters which has been transmitted through and/or reflected from breast tissue and received by the light detectors can be analyzed to detect and/or image abnormal breast tissue. In an example, the air-gap-reducing layer can be transparent. In an example, the air-gap-reducing layer can have the same values for one or more optical parameters as normal breast tissue.

In an example, expandable components can be expanded by being filled with a gas. In an example, expandable components can be expanded by being filled with a liquid. In an example, expandable components can be expanded by electromagnetic actuators. In an example, expandable components can be individually and selectively expanded so that they are configured to compress wider portions of the breast to a greater degree than narrower portions of the breast. In an example, expandable components which are farther from the virtual plane can be expanded more than expandable components which are closer to the virtual plane.

In an example, the device can further comprise marks, indicators, openings, or sensors which are configured to help register and/or align the device relative to the anatomy of the breast. In an example, the device can further comprise other components selected from the group consisting of: power source, data processor, wireless data transmitter, wireless data receiver, air pump, and liquid pump. In an example, one or more of these other components can be located on the back strap of a bra. Relevant design variations discussed in priority-linked disclosures can also be applied to the example shown here.

I claim:

1. A multi-layer device for optical detection of breast cancer comprising:
an air-gap-reducing layer which is configured to be worn on the surface of a person's breast, wherein the air-gap-reducing layer is transparent or has the same optical characteristics as normal breast tissue, and wherein a first part of the air-gap-reducing layer is on a first side of a virtual plane and a second part of the air-gap-reducing layer is on a second side of the virtual plane;
an optical layer with a plurality of light emitters and light detectors, wherein a first part of the optical layer is on the first side of the virtual plane and a second part of the optical layer is on the second side of the virtual plane;
an expandable layer with a plurality of expandable components, wherein a first part of the expandable layer is on the first side of the virtual plane and a second part of the expandable layer is on the second side of the virtual plane; and
a structural layer which reduces expansion of the expandable components away from the breast; wherein the optical layer is between the air-gap-reducing layer and the expandable layer, wherein the expandable layer is between the optical layer and the structural layer, wherein the device has an unexpanded configuration in which the expandable components are not expanded, wherein the device has an expanded configuration in which the expandable components are expanded, wherein there is a first average distance between the first part of the optical layer and the second part of the optical layer when the device is in the unexpanded configuration, wherein there is a second average distance between the first part of the optical layer and the second part of the optical layer when the device is in the expanded configuration, and wherein the second average distance is less than the first average distance.

2. The device in claim 1 wherein the device is a bra.

3. The device in claim 1 wherein the device is inserted into a bra cup.

4. The device in claim 1 wherein the device is configured to be worn between a bra cup and a breast.

5. The device in claim 1 wherein the device is an adhesive patch, sticker, or bandage.

6. The device in claim 1 wherein the device has a teardrop-shaped perimeter.

7. The device in claim 6 wherein the teardrop shape has an apex and/or vertex and wherein the apex and/or vertex is configured to be placed over the Auxiliary Tail of Spence.

8. The device in claim 1 wherein the virtual plane is an oblique virtual plane and wherein the oblique virtual plane is configured to span from the upper outer quadrant to the lower inner quadrant of the breast.

9. The device in claim 1 wherein there are light emitters on the first side of the virtual plane and light detectors on the second side of the virtual plane.

10. The device in claim 1 wherein light from the light emitters which has been transmitted through and/or reflected from breast tissue and received by the light detectors is analyzed to detect and/or image abnormal breast tissue.

11. The device in claim 1 wherein the air-gap-reducing layer is transparent.

12. The device in claim 1 wherein the air-gap-reducing layer has the same values for one or more optical parameters as normal breast tissue.

13. The device in claim 1 wherein expandable components are expanded by being filled with a gas.

14. The device in claim 1 wherein expandable components are expanded by being filled with a liquid.

15. The device in claim 1 wherein expandable components are expanded by electromagnetic actuators.

16. The device in claim 1 wherein expandable components are individually and selectively expanded so that they are configured to compress wider portions of the breast to a greater degree than narrower portions of the breast.

17. The device in claim 1 wherein expandable components which are farther from the virtual plane are expanded more than expandable components which are closer to the virtual plane.

18. The device in claim 1 wherein the device further comprises marks, indicators, openings, or sensors which are configured to help register and/or align the device relative to anatomy of the breast.

19. The device in claim 1 wherein the device further comprises other components selected from the group consisting of: power source, data processor, wireless data transmitter, wireless data receiver, air pump, and liquid pump.

20. The device in claim 19 wherein one or more of the other components are located on the back strap of a bra.
